(19) United States
(12) Patent Application Publication
(10) Pub. No.: US 2026/0060936 A1
(43) Pub. Date: Mar. 5, 2026

(54) INDOLIUM-BASED STABILIZERS OF HYDROPHOBIC DRUGS

(71) Applicant: TECHNION RESEARCH & DEVELOPMENT FOUNDATION LTD., Haifa (IL)

(72) Inventors: Yosi SHAMAY, Haifa (IL); Yuval HARRIS, Haifa (IL); Danna NIEZNI, Haifa (IL); Hagit SASON, Haifa (IL)

(21) Appl. No.: 19/104,171

(22) PCT Filed: Aug. 29, 2023
(86) PCT No.: PCT/IL2023/050916

§ 371 Date: Feb. 14, 2025

Related U.S. Application Data
(60) Provisional application No. 63/374,033, filed on Aug. 31, 2022.

(51) Int. Cl. A61K 9/51; A61K 31/337; A61K 31/4166; A61K 31/436; A61K 31/454; A61K 31/506; A61K 31/519; A61K 31/5377; A61K 31/557; A61K 31/635; A61K 38/13; A61K 38/20; A61K 38/21; A61K 45/06; A61K 47/22

(52) U.S. Cl. CPC A61K 9/5123; A61K 31/337; A61K 31/4166; A61K 31/436; A61K 31/454; A61K 31/506; A61K 31/519; A61K 31/5377; A61K 31/557; A61K 31/635; A61K 38/13; A61K 38/20; A61K 38/21; A61K 45/06; A61K 47/22

(57) ABSTRACT

A composition containing a mixture of oligomers or co-oligomers obtained by exposing an indolium-based monomer, optionally together with an additional monomer selected from dopamine, L-dopa, norepinephrine, serotonin, and a mixture thereof, to a basic buffer. The composition may further contain a hydrophobic substance such as a drug or dietary supplement, stabilized by the oligomers above.

### TECHNICAL FIELD

The present invention relates to a composition comprising a mixture of oligomers or co-oligomers obtained by exposing an indolium-based monomer, to a basic buffer, which may further comprise a hydrophobic substance such as a drug or dietary supplement, stabilized by said oligomers above.

### BACKGROUND ART

Many small molecule drugs are poorly soluble in water and form large colloidal aggregates, which can reduce their bioavailability, route of administration, and therapeutic effectiveness. In nanomedicine, drug loaded nanoparticles are usually stabilized with biomaterials based on small molecules such as lipids, and synthetic or biological polymers like PLGA or a fucoidan, preventing aggregation and improving their shelf life. These fabrication processes are limited by the fact that they usually have low drug loading and lack predictability. Recently, it has been shown that co-precipitating hydrophobic drugs with small molecule dyes, such as a Congo red and IR783, can produce stable nanoparticles with ultra-high drug loading of up to 85%, similar or equivalent to drug nanocrystals (Shamay et al., 2018). Furthermore, a predictive algorithm was developed to identify which drugs can be formulated with this protocol revealing that only 4% of the small molecule drug space can use IR783 as a stabilizer (Shamay et al., 2018). Moreover, it was shown that within the 4% prediction space, some of the resulting nanoparticles were unstable which can limit their translation into clinical use.

Recent development of algorithms and automation processes of liquid handling and control systems have allowed the accelerated discovery of both novel materials and chemical reactions. Some refer to these systems as chemical robots as they are controllable instruments performing chemistry. According to the Cronin lab, the most advanced system so far is the Chemputation project, which claims to fully automate any published chemical product. In order to allow intelligent chemical robots with automated decision-making, a clear quantifiable readout of success or failure must be incorporated.

Polydopamine (PDA) is highly popular in biomedical research due to its simple preparation, good biocompatibility, and versatility in bioconjugation. Although the specific synthetic mechanism and structure of PDA auto-oxidative polymerization in alkaline buffers has not been clarified, it was found to be useful in many fields including biomedicine, where it had attracted significant interest in the field of tumor targeted drug delivery due to its photothermal abilities, nanoparticle (NP) coating and drug encapsulation via pi-pi stacking.

Compared to other drug delivery systems (DDS), PDA has a few advantages mainly because of the simple and mild conditions required for its synthesis (e.g., no organic solvent is required). In addition, due to its superior adhesion, PDA is capable of coating various types of organic and inorganic nanoparticles. Furthermore, the catechol and anthracene fractions found on PDA surface allow secondary modification with thiol or amino group-constraining compounds, through Michael addition reaction or Schiff base reaction under alkaline condition (Shamay et al., 2018).

Though as a homopolymer it is incredibly useful for coating existing materials, it is rarely used alone as a DDS. The most common post polymerization modification for PDA based systems is PEGylation which was used, e.g., for delivery of doxorubicin and hydroxycamptothecin with high encapsulation efficiency.

Rather than post polymerization modifications, one-step methods have been proposed for the preparation of PDA copolymers with monomers containing catechol and either acrylate or amine groups. An interesting example is polydopamine-polyethyleneimine (PDA-PEI) copolymer, which has intrinsic fluorescence that is orders of magnitude more efficient than PDA and melanin. However, while most of the publications propose PDA shells loaded with a drug, PDA copolymers have not been studied in the context of drug nanoprecipitation. In this method, the drug is dissolved in an organic solvent, which is added gradually to an aqueous solution, and nanoparticles are formed spontaneously in the presence of a stabilizer, resulting in a high encapsulation ratio of the drug in the nanoparticles.

### SUMMARY OF INVENTION

In one aspect, disclosed herein is a composition comprising a mixture of oligomers obtained by exposing an indolium-based monomer of formula I:

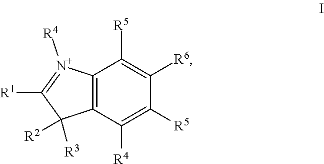

wherein:
R¹, R², and R³ each independently is (C₁-C₆)alkyl optionally interrupted with one or more heteroatoms selected from O, N, and S;
R⁴ is (C₁-C₁₂)alkyl optionally interrupted with one or more heteroatoms selected from O, N, and S, substituted with one or more groups each independently selected from —SO₃⁻, —COO⁻, —PO₃⁻², —OH, —NH₂, —N⁺(R′)₃, phosphocholine (—OPO₃⁻—(CH₂)₂—N⁺(CH₃)₃), and acetoxyethyl phospocholine (—CH₂—C(O)—O—(CH₂)₂—OPO₃⁻—(CH₂)₂—N⁺(CH₃)₃), wherein R′ each independently is (C₁-C₆)alkyl, or (C₃-C₇)cycloalkyl, or two of the R's together with the nitrogen atom to which they are attached form a 5-9 membered ring;
R⁵, R⁶, R⁷, and R⁸ each independently is selected from H, —OH, (C₁-C₆)alkyl, —O—(C₁-C₆)alkyl, —CO(C₁-C₆)alkyl, —COO(C₁-C₆)alkyl, —N(R⁹)₂, —CON(R⁹)₂, —S—(C₁-C₆)alkyl, and —SH; or two adjacent of R⁵, R⁶, R⁷, and R⁸ (i.e., R⁵ and R⁶, R⁶ and R⁷, or R⁷ and R⁸) together with the carbon atoms to which they are attached form (C₆-C₁₄)aryl or 5- to 14-membered heteroaryl, optionally substituted by one or more groups each independently selected from halogen, —OH, (C₁-C₆)alkyl, —O—(C₁-C₆)alkyl, —CO(C₁-C₆)alkyl, —COO(C₁-C₆)alkyl, —N(R₉)₂, —CON(R₉)₂, —S—(C₁-C₆)alkyl, and —SH, and the other of R⁵, R⁶, R⁷, and R⁸ each independently is selected from H, —OH, (C₁-C₆)alkyl, —O—(C₁-C₆)alkyl, —CO(C₁-C₆)alkyl, —COO(C₁-C₆)alkyl, —N(R₉)₂, —CON(R⁹)₂, —S—(C₁-C₆)alkyl, and —SH; and
R⁹ each independently is H, halogen, (C₁-C₆)alkyl, (C₃-C₁₁)cycloalkyl, (C₅-C₁₁)cycloalkenyl, heterocyclyl, aryl, heteroaryl, or the two R⁹'s together with the nitrogen atom to which they are attach form a 5- to 7-membered ring,
optionally together with an additional monomer selected from dopamine, L-dopa, norepinephrine, serotonin, and a mixture thereof, to a basic buffer.

In certain embodiments, the composition disclosed herein comprises a mixture of oligomers obtained by exposing an indolium-based monomer of the formula I as defined above, together with an additional monomer selected from dopamine, L-dopa, norepinephrine, serotonin, and a mixture thereof, to a basic buffer. In other embodiments, said composition comprises a mixture of oligomers obtained by exposing an indolium-based monomer of the formula I as defined above, without said additional monomer, i.e., in the absence of said additional monomer, to a basic buffer.

In a particular such aspect, the composition disclosed herein as defined in any one of the embodiments above further comprises a hydrophobic substance, e.g., a hydrophobic drug or dietary supplement, that is stabilized by said oligomers above. Such a composition is referred to herein as “a hydrophobic substance-containing composition”).

In another aspect, disclosed herein is a method for identifying whether a compound or mixture of compounds is capable of stabilizing (both in storage and in vivo) a hydrophobic substance such as a hydrophobic drug and dietary supplement, said hydrophobic substance exhibiting a fluorescence emission in the aggregated state that is significantly increased compared to its fluorescence emission in the non-aggregated, i.e., soluble, state, said method comprising the steps of:
(i) mixing an aqueous solution of said compound or mixture of compounds with said hydrophobic substance to obtain a suspension of nanoparticles each comprising said compound or mixture of compounds and said hydrophobic substance;
(ii) imaging the fluorescence emission of the suspension obtained; and
(iii) comparing said fluorescence emission with the fluorescence emission of said hydrophobic substance in its aggregated state optionally divided by white light (brightfield),
wherein a significant decrease, e.g., a decrease of at least about 20%, 30%, 40%, 50%, 60%, 70%, 80%, 90%, 95%, or more, in the fluorescence emission of said substance in said suspension compared to its fluorescence emission in the aggregated state optionally divided by white light (brightfield) indicates that said compound or mixture of compounds is capable of stabilizing said substance.

### BRIEF DESCRIPTION OF DRAWINGS

FIGS. 1A-1F show identification and characterization of AIEgen drugs. (1A) Emission spectrum intensities of various anti-cancer drugs dissolved in DMSO (upper panel) or precipitated with the addition of DDW (lower panel). (ex.320 nm). (1B) Max fluorescence intensity subtraction of drugs in the dissolved state from the solid state, exhibiting AIE or ACQ activity. (1C) Examples of full excitation and emission spectrums of selected drugs dissolved vs. precipitated. (1D) Automated fluorescent microscopic images (ex.377 nm em.447 nm) of aggregated drugs revealing different sedimentation microstructures/morphologies. (1E) Quantification of total emission intensity of the drugs listed 1B as well as several additional drugs (afatinib, bicalutamide, celecoxib, cyclophosphamide, dasatinib, defactinib, paclitaxel, tanespimycin, thalidomide, and vorinostat), all shown with abbreviated names, in the microscopic images (the total emission intensity of alpelisib and gefitinib listed in 1B, as well as of carfilzomib, olaparib and sunitinib, was about zero and these drugs are therefore not shown). (1F) Macromolecular drug aggregates under white light or UV lamp (365 nm) in the dissolved state (DMSO), as part of nanoparticles (INP), and in acetonitrile-disassembled nanoparticles (INP+ACN).

FIGS. 2A-2G show identification of ultra-stabilizer R595 using automated synthesis, self-assembly, and AIE microscopy. (2A) Workflow of the automated process to discover new drug stabilizers in a 96 well plate using a liquid handling robot and automatic fluorescent microscopy. (2B) Synthesized products after 24 h of mixing in white light (left) and UV light (middle). Right-center wavelength from absorbance spectrum of the products. (2C) Left-Products and trametinib mixtures under UV light. Quantification of the sum area of light (brightfield, upper right) and fluorescence (AIE, lower left) intensity using image analysis, and the brightfield/DAPI ratio (lower right). Dashed line indicates lead stabilizer candidates; (2D) Fluorescence excitation and emission spectrum of R595. (2E) ¹H-NMR spectrum of the monomer indol820 and R595. (2F) DLS measurement of trametinib and nilotinib-R595 nanoparticles. (2G) HR-SEM image of trametinib-R595 nanoparticles. Scale bar=100 nm.

FIGS. 3A-3E show that R595 is an ultra-stabilizer. (3A) Result of attempted water dispersion of nilotinib with a panel of known stabilizers. Pluronic F127, SDS, carboxymethyl cellulose (CMC), α-cyclodextrin (αCD), β-cyclodextrin (βCD), β-cyclodextrin sulfobutyl ether (βCD-SO₃), and HSA. Arrows indicate drug aggregation. (3B) Size measurements of nanoparticles composed of small molecule drugs and dyes (left panel) and their corresponding PDI (right panel). (3C) Aggregation kinetic profiles of nanoparticles in the DAPI channel of a fluorescent microscope. (3D) Size measurements over time of trametinib nanoparticles with different dyes (left panel) and their corresponding PDI (right panel). (3E) Size measurements of nanoparticles of nilotinib and carfilzomib with R595 or IR783 (left panel) and their corresponding PDI (right panel). Error bars indicate mean±s.d. * p<0.05, ** p<0.005, *** p<0.0005, **** p<0.00005 by unpaired t-test.

FIGS. 4A-4F show in vitro uptake and efficacy of R595 nanoparticles. (4A) Representative fluorescence images of various cell lines incubated for 3 h with R595 nanoparticles. Scale bar=50 μm. (4B) Quantification of the mean fluorescence intensity in each cell line. (4C) Representative microscopic images of KPL spheroids incubated with nanoparticles, blue-drugs. Scale bar=100 μm. (4D) KPL (upper panel) and HCT116 (lower panel) cell viability assay with 7 different drugs. (4E) KPL and HCT116 3D viability assay comparing free trametinib (FD) with trametinib R595 nanoparticles. (4F) Anti-tumor efficacy in HCT116 spheroids as measured with diameter change over time after incubation with 6×10⁻⁹ mg/ml trametinib (FD) or trametinib-R595 nanoparticles. Error bars indicate mean±s.d. * p<0.05, *** p<0.0005, **** p<0.00005 by unpaired t-test.

FIGS. 5A-5G show in vivo biodistribution, efficacy and safety of trametinib-R595 nanoparticles. (5A) Biodistribution of trametinib-R595 nanoparticles 24 h after IP injection to HCT116 tumor xenografts model as measured with IVIS (ex. 600 nm, em. 660). (5B) In vivo efficacy measured by % of tumor volume from day of randomization. The two days of treatment are marked in black arrows. (5C) Body weight change from t0 of tumor-bearing mice treated with either nanoparticles or free trametinib. (5D) Survival curve of mice in the HCT116 experiment. (5E) Survival curve of mice in the lung metastases KPL model. (5F) Body weight change from day0 of the KPL model. (5G) Representative images of H&E staining in different tissues of mice treated bi-weekly with nanoparticles compared with control. Error bars indicate mean±s.d. * p<0.05, ** p<0.005, *** p<0.0005, **** p<0.00005 by unpaired t-test.

FIGS. 6A-6B show additional nanoparticles made with hydrophobic drugs, sorafenib (sor) and trametinib (tra), and tested for size (6A), and PDI in day 1 at 3 days (6B). The indoliums were tested alone and together in a 1:1 mass ratio.

FIGS. 7A-7D show polymerization process. (7A) Photograph of different polymerization conditions and their impact on the polymerization of catecholamines after 4.5 hr. DA=dopamine, DO=L-dopa, NE=norepinephrine, SE=serotonin, TY=tyramine, Tryp=tryptamine. (7B) Absorbance spectra of dopamine/PDA and L-dopa/PDO in bicarbonate and Tris buffer. (7C) ATR spectra of dopamine/PDA and L-dopa/PDO in bicarbonate. (7D) Absorbance spectra of the reactions over time in bicarbonate buffer (0.1M) and their structures from 0 h until 4.5 h.

FIGS. 8A-8D show selection of top copolymer. (8A) Left—co-monomers matrix, illustration of colors after a 4 hr reaction in sodium bicarbonate buffer 0.1M. Right—normalized confluence of blue emission at 477 nm with nilotinib. (8B) Average diameter of sorafenib and nilotinib nanoparticles formulated with different polymers. (8C) PDI of sorafenib and nilotinib nanoparticles made with different copolymers. (8D) Stabilization over-time of nilotinib nanoparticles formulated with different stabilizers.

FIG. 9 shows three-day stability of 9 drugs stabilized by PDA-In820 (upper panel), PDA-PDO-In820 (middle panel), and PDO-820 (lower panel).

FIG. 10 shows HR-SEM images of sorafenib nanoparticles formulated with PDA-PDO-In820, PDA-In820, and PDO-In820. Scale bar=200 nm.

FIGS. 11A-11C show characterization and optimization of PDA-PDO-In820. (11A) Nilotinib nanoparticles formulated during different periods of time since polymerization initiation. (11B) Encapsulation ratios of nilotinib nanoparticles from 10A. (11C) Size of ponatinib nanoparticles formulated in different time points during the aging process of PDA-PDO-In820.

FIG. 12 shows in-vitro toxicity assay in mouse embryonic fibroblasts, 3T3 cells. Image-based cell counting at various times of incubation with PDA-In820 (upper panel), PDO-In820 (middle panel), and PDA-PDO-In820 (lower panel).

FIG. 13 shows images of cell morphologies at various concentrations incubated with the different copolymers. Scale bar=100 μm.

FIGS. 14A-14C show in-vitro toxicity assay of enzalutamide nanoparticles in mouse embryonic fibroblasts, 3T3 cells. (14A) Image-based cell counting at various times of incubation with free enzalutamide. (14B) MTT after 3 days of incubation with free enzalutamide and its nanoparticles. (14C) Image-based cell counting at various times of incubation with the enzalutamide nanoparticles based on PDA-In820 (upper panel), PDO-In820 (middle panel), and PDA-PDO-In820 (lower panel).

FIG. 15 shows images of cell morphologies at various concentrations incubated with nanoparticles of the different copolymers and free enzalutamide. Scale bar=100 μm.

FIGS. 16A-16E show cell viability assays in 2D and 3D models of HCT116. (16A) HR-SEM images of the trametinib and sorafenib nanoparticles stabilized by PDA-PDO-In820, scale bar=100 nm. (16B) CTG 2D results of HCT116 cells after 4 days of treatment (p<0.05). (16C) CTG 3D results of HCT116 spheroids after 5 days of treatment (p<0.05). (16D) Relative change in spheroids size on the 2^{nd} and 5^{th} days of treatment. (16E) Spheroids images on the 1^{st}, 2^{nd} and 5^{th} days of the treatment. drugs concentration=10⁻³ mg/ml, scale bar=500 μm.

### DETAILED DESCRIPTION

It has now been found, in accordance with the present invention, that by exposing an indolium-based monomer such as 1,1,2-trimethyl-3-(4-sulfobutyl)benz[e]indolium inner salt and 2,3,3-trimethyl-1-(4-sulfobutyl)indolium inner salt to a basic buffer such as sodium bicarbonate buffer, a mixture of oligomers capable of stabilizing hydrophobic drugs such as nilotinib and trametinib in an aqueous medium is obtained. As shown herein, nanoparticles made of 1,1,2-trimethyl-3-(4-sulfobutyl)benz[e]indolium inner salt and trametinib were stable for a period of six months, exhibiting constant polydispersity index (PDI) and size over time. Interestingly, such nanoparticles, when injected IP to HCT116 subcutaneous colorectal cancer xenograft mouse model, had higher anticancer efficacy compared with the free drug given orally.

As further found, by exposing 1,1,2-trimethyl-3-(4-sulfobutyl)benz[e]indolium inner salt together with a mixture of dopamine and L-dopa to a basic buffer such as sodium bicarbonate buffer, a non-toxic and safe stabilizer, as shown in fibroblasts in vitro, is obtained.

In one aspect, the present invention thus provides a composition comprising a mixture of oligomers obtained by exposing an indolium-based monomer of formula I, optionally together with an additional monomer selected from dopamine, L-dopa, norepinephrine, serotonin, and a mixture thereof, to a basic buffer,

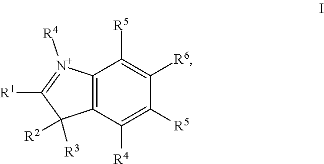

wherein:
R¹, R², and R³ each independently is (C₁-C₆)alkyl optionally interrupted with one or more heteroatoms selected from O, N, and S;
R⁴ is (C₁-C₁₂)alkyl optionally interrupted with one or more heteroatoms selected from O, N, and S, substituted with one or more groups each independently selected from —SO₃⁻, —COO⁻, —PO₃⁻², —OH, —NH₂, —N⁺(R′)₃, phosphocholine (—OPO₃⁻—(CH₂)₂—N⁺(CH₃)₃), and acetoxyethyl phospocholine (—CH₂—C(O)—O—(CH₂)₂—OPO₃⁻—(CH₂)₂—N⁺(CH₃)₃), wherein R′ each independently is (C₁-C₆)alkyl, or (C₃-C₇)cycloalkyl, or two of the R's together with the nitrogen atom to which they are attached form a 5-9 membered ring;
R⁵, R⁶, R⁷, and R⁸ each independently is selected from H, —OH, (C₁-C₆)alkyl, —O—(C₁-C₆)alkyl, —CO(C₁-C₆)alkyl, —COO(C₁-C₆)alkyl, —N(R⁹)₂, —CON(R⁹)₂, —S—(C₁-C₆)alkyl, and —SH; or two adjacent of R⁵, R⁶, R⁷, and R⁸ (i.e., R⁵ and R⁶, R⁶ and R⁷, or R⁷ and R⁸) together with the carbon atoms to which they are attached form (C₆-C₁₄)aryl or 5- to 14-membered heteroaryl, optionally substituted by one or more groups each independently selected from halogen, —OH, (C₁-C₆)alkyl, —O—(C₁-C₆)alkyl, —CO(C₁-C₆)alkyl, —COO(C₁-C₆)alkyl, —N(R₉)₂, —CON(R₉)₂, —S—(C₁-C₆)alkyl, and —SH, and the other of R⁵, R⁶, R⁷, and R⁸ each independently is selected from H, —OH, (C₁-C₆)alkyl, —O—(C₁-C₆)alkyl, —CO(C₁-C₆)alkyl, —COO(C₁-C₆)alkyl, —N(R₉)₂, —CON(R⁹)₂, —S—(C₁-C₆)alkyl, and —SH; and
R⁹ each independently is H, halogen, (C₁-C₆)alkyl, (C₃-C₁₁)cycloalkyl, (C₅-C₁₁)cycloalkenyl, heterocyclyl, aryl, heteroaryl, or the two R⁹'s together with the nitrogen atom to which they are attach form a 5- to 7-membered ring.

The term “alkyl” typically means a linear or branched hydrocarbon group having, e.g., 1-12 carbon atoms and includes methyl, ethyl, n-propyl, isopropyl, n-butyl, sec-butyl, isobutyl, tert-butyl, n-pentyl, 2,2-dimethylpropyl, n-hexyl, n-heptyl, n-octyl, n-nonyl, n-decyl, n-undecyl, n-dodecyl, and the like. Preferred are (C₁-C₆)alkyl, more preferred (C₁-C₄)alkyl, groups, i.e., methyl, ethyl, n-propyl, isopropyl, n-butyl, sec-butyl, isobutyl, and tert-butyl.

The term “cycloalkyl” means a mono- or bicyclic saturated hydrocarbyl group having, e.g., 3-11, but preferably 3-7, carbon atoms such as cyclopropyl, cyclobutyl, cyclopentyl, cyclohexyl, cycloheptyl, cyclooctyl, and the like, that may be substituted, e.g., by one or more alkyl groups. The term “cycloalkenyl” means a mono- or bicyclic hydrocarbyl group having, e.g., 3-11 carbon atoms and one or more double bonds such as cyclopropenyl (e.g., 2-cyclopropen-1-yl), cyclobutenyl (e.g., 2-cyclobuten-1-yl), cyclopentenyl (e.g., 2-cyclopenten-1-yl, and 3-cyclopenten-1-yl), cyclohexenyl (e.g., 2-cyclohexen-1-yl, and 3-cyclohexen-1-yl), and the like, that may be substituted, e.g., by one or more alkyl groups.

The term “heterocyclic ring” as used herein denotes a mono-, bi-, or poly-cyclic non-aromatic ring of, e.g., 3-12 atoms, consisting of at least one carbon atom and at least one heteroatom selected from oxygen, nitrogen, and sulfur (optionally oxidized), which may be saturated or unsaturated, i.e., containing at least one unsaturated bond. Preferred are 5- or 6-membered heterocyclic rings. The heterocyclic ring may be substituted, e.g., by one or more alkyl groups, and may also be oxidized at either a carbon atom or a heteroatom thereof (in cases said heteroatom is sulfur, it may also be dioxidized). The term “heterocyclyl” as used herein refers to any univalent group derived from a heterocyclic ring as defined herein by removal of hydrogen atom from any of the ring atoms. Examples of such groups include, without limitation, pyridinyl, pyrimidinyl, aziridinyl, piperidinyl, pyrrolidinyl, azepinyl, morpholinyl such as 4-morpholinyl, oxazolyl, dihydrooxazolyl, oxadiazolyl; imidazolyl, imidazolinyl, dihydroimidazolyl, pyrazolyl, triazolyl, tetrazolyl, thiadiazolyl, piperazinyl, tetrahydropirydinyl, and oxapinyl.

The term “aryl” denotes an aromatic carbocyclic group having, e.g., 6-14 carbon atoms and consisting of a single ring or multiple rings either condensed or linked by a covalent bond. Examples of such groups include, without being limited to, phenyl, naphthyl, phenanthryl, and biphenyl. The aryl may optionally be substituted by one or more groups each independently selected from halogen, —OH, (C₁-C₆)alkyl, —O—(C₁-C₆)alkyl, —CO(C₁-C₆)alkyl, —COO(C₁-C₆)alkyl, —N(R₉)₂, —CON(R₉)₂, —S—(C₁-C₆)alkyl, and —SH.

The term “heteroaromatic ring” as used herein refers to a mono-, bi-, or poly-cyclic aromatic ring having, e.g., 5-14 atoms, and consisting of at least one carbon atom and at least one, preferably 1-2, heteroatom each independently selected from oxygen, nitrogen, and sulfur (optionally oxidized).

The term “heteroaryl” refers to a group derived from a heteroaromatic ring as defined herein by removal of hydrogen atom from any of the ring atoms. Examples of mono-cyclic heteroaryls include, without being limited to, pyrrolyl, furyl, thienyl, thiazinyl, pyrazolyl, pyrazinyl, imidazolyl, oxazolyl, isoxazolyl, thiazolyl, isothiazolyl, pyridyl, pyrimidinyl, 1,2,3-triazinyl, 1,3,4-triazinyl, and 1,3,5-triazinyl. Polycyclic heteroaryl radicals are preferably composed of two rings such as, but not limited to, benzofuryl, isobenzofuryl, benzothienyl, indolyl, quinolinyl, isoquinolinyl, imidazo[1,2-a]pyridyl, benzimidazolyl, benzthiazolyl, benzoxazolyl, pyrido[1,2-a]pyrimidinyl and 1,3-benzodioxinyl. The heteroaryl may optionally be substituted by one or more groups each independently selected from halogen, —OH, (C₁-C₆)alkyl, —O—(C₁-C₆)alkyl, —CO(C₁-C₆)alkyl, —COO(C₁-C₆)alkyl, —N(R₉)₂, —CON(R₉)₂, —S—(C₁-C₆)alkyl, and —SH. It should be understood that when a polycyclic heteroaryl is substituted, the substitution may be in any of the carbocyclic and/or heterocyclic rings.

The term “halogen” as used herein refers to a halogen and includes fluoro, chloro, bromo, and iodo, but it is preferably chloro or bromo.

In certain embodiments, disclosed herein is a composition comprising a mixture of oligomers as defined above, wherein R¹, R², and R³ each independently is (C₁-C₆)alkyl, preferably methyl, ethyl, propyl, or isopropyl. Particular such embodiments are those wherein R¹, R², and R³ are identical, e.g., wherein R¹, R², and R³ each is methyl or ethyl.

In certain embodiments, disclosed herein is a composition comprising a mixture of oligomers as defined above, wherein R⁴ is (C₁-C₆)alkyl substituted with one or more —SO₃, phosphocholine, or acetoxyethyl phospocholine groups, preferably with a single —SO₃⁻, phosphocholine, or acetoxyethyl phospocholine group. Particular such embodiments are those wherein R⁴ is a linear (C₁-C₆)alkyl substituted with a sole —SO₃⁻, phosphocholine, or acetoxyethyl phospocholine group at the omega (terminal) position thereof, i.e., a group of the formula —(CH₂)₁₋₆—SO₃⁻, e.g., wherein R⁴ is a group of the formula —(CH₂)₄—SO₃⁻, —(CH₂)₃—SO₃⁻, —(CH₂)₂—SO₃⁻, or —(CH₂)₁—SO₃⁻; a group of the formula —(CH₂)₁₋₆—OPO₃⁻—(CH₂)₂—N⁺(CH₃)₃, e.g., wherein R⁴ is a group of the formula —(CH₂)₄—OPO₃⁻—(CH₂)₂—N⁺(CH₃)₃, —(CH₂)₃—OPO₃⁻—(CH₂)₂—N⁺(CH₃)₃, —(CH₂)₂—OPO₃⁻—(CH₂)₂—N⁺(CH₃)₃, or —CH₂—OPO₃⁻—(CH₂)₂—N⁺(CH₃)₃; or a group of the formula —(CH₂)₁₋₆—CH₂—C(O)—O—(CH₂)₂—OPO₃⁻—(CH₂)₂—N⁺(CH₃)₃, e.g., wherein R⁴ is a group of the formula —(CH₂)₄—C(O)—O—(CH₂)₂—OPO₃⁻—(CH₂)₂—N⁺(CH₃)₃, —(CH₂)₃—C(O)—O—(CH₂)₂—OPO₃⁻—(CH₂)₂—N⁺(CH₃)₃, or —(CH₂)₂—C(O)—O—(CH₂)₂—OPO₃⁻—(CH₂)₂—N⁺(CH₃)₃.

In certain embodiments, disclosed herein is a composition comprising a mixture of oligomers as defined above, wherein R⁵, R⁶, R⁷, and R⁸ each independently is H, or (C₁-C₆)alkyl such as (C₁-C₃)alkyl; or R⁵ and R⁶ each independently is H, or (C₁-C₆)alkyl such as (C₁-C₃)alkyl, and R⁷ and R⁸ together with the carbon atoms to which they are attached form an optionally substituted (C₆-C₁₄)aryl, e.g., phenyl; or R⁵ and R⁸ each independently is H, or (C₁-C₆)alkyl such as (C₁-C₃)alkyl, and R⁶ and R⁷ together with the carbon atoms to which they are attached form an optionally substituted (C₆-C₁₄)aryl, e.g., phenyl; or R⁷ and R⁸ each independently is H, or (C₁-C₆)alkyl such as (C₁-C₃)alkyl, and R⁵ and R⁶ together with the carbon atoms to which they are attached form an optionally substituted (C₆-C₁₄)aryl, e.g., phenyl. In particular such embodiments, R⁵, R⁶, R⁷, and R⁸ each is H; or R⁵ and R⁶ each is H, and R⁷ and R⁸ together with the carbon atoms to which they are attached form an optionally substituted (C₆-C₁₄)aryl, e.g., phenyl; or R⁵ and R⁸ each is H, and R⁶ and R⁷ together with the carbon atoms to which they are attached form an optionally substituted (C₆-C₁₄)aryl, e.g., phenyl; or R⁷ and R⁸ each is H, and R⁵ and R⁶ together with the carbon atoms to which they are attached form an optionally substituted (C₆-C₁₄)aryl, e.g., phenyl.

In certain embodiments, disclosed herein is a composition comprising a mixture of oligomers as defined above, wherein R¹, R², and R³ each independently is (C₁-C₆)alkyl, preferably methyl, ethyl, propyl, or isopropyl; R⁴ is (C₁-C₆)alkyl substituted with one or more, but preferably one, —SO₃⁻, phosphocholine, or acetoxyethyl phospocholine groups; and: (i) R⁵, R⁶, R⁷, and R⁸ each independently is H, or (C₁-C₆)alkyl such as (C₁-C₃)alkyl; (ii) R⁵ and R⁶ each independently is H or (C₁-C₆)alkyl such as (C₁-C₃)alkyl, and R⁷ and R⁸ together with the carbon atoms to which they are attached form an optionally substituted (C₆-C₁₄)aryl, e.g., phenyl; (iii) R⁵ and R⁸ each independently is H, or (C₁-C₆)alkyl such as (C₁-C₃)alkyl, and R⁶ and R⁷ together with the carbon atoms to which they are attached form an optionally substituted (C₆-C₁₄)aryl, e.g., phenyl; or (iv) R⁷ and R⁸ each independently is H, or (C₁-C₆)alkyl such as (C₁-C₃)alkyl, and R⁵ and R⁶ together with the carbon atoms to which they are attached form an optionally substituted (C₆-C₁₄)aryl, e.g., phenyl. In particular such embodiments, R¹, R², and R³ are identical; R⁴ is a linear (C₁-C₆)alkyl substituted with a sole —SO₃⁻, phosphocholine, or acetoxyethyl phospocholine group at the omega (terminal) position thereof; R⁵ and R⁶ each is H; and R⁷ and R⁸ together with the carbon atoms to which they are attached form phenyl. In more particular such embodiments, R¹, R², and R³ each is methyl; R⁴ is —(CH₂)₄—SO₃⁻, —(CH₂)₃—SO₃⁻, —(CH₂)₂—SO₃⁻, —(CH₂)₁—SO₃⁻, —(CH₂)₄—OPO₃⁻—(CH₂)₂—N⁺(CH₃)₃, —(CH₂)₃—OPO₃⁻—(CH₂)₂—N⁺(CH₃)₃, —(CH₂)₂—OPO₃⁻—(CH₂)₂—N⁺(CH₃)₃, or —CH₂—OPO₃⁻—(CH₂)₂—N⁺(CH₃)₃, —(CH₂)₄—C(O)—O—(CH₂)₂—OPO₃⁻—(CH₂)₂—N⁺(CH₃)₃, —(CH₂)₃—C(O)—O—(CH₂)₂—OPO₃⁻—(CH₂)₂—N⁺(CH₃)₃, or —(CH₂)₂—C(O)—O—(CH₂)₂—OPO₃⁻—(CH₂)₂—N⁺(CH₃)₃; and either: (i) R⁵, R⁶, R⁷, and R⁸ each is H; or (ii) R⁵ and R⁶ each is H; and R⁷ and R⁸ together with the carbon atoms to which they are attached form phenyl.

In certain specific embodiments, R¹, R², and R³ each is methyl; R⁴ is —(CH₂)₄—SO₃; and either: (i) R⁵, R⁶, R⁷, and R⁸ each is H (2,3,3-trimethyl-1-(4-sulfobutyl)indolium inner salt, also referred to herein as In783); or (ii) R⁵ and R⁶ each is H; and R⁷ and R⁸ together with the carbon atoms to which they are attached form phenyl (1,1,2-trimethyl-3-(4-sulfobutyl)benz[e]indolium inner salt, also referred to herein as In820). In other specific embodiments, R¹, R², and R³ each is methyl; R⁴ is —(CH₂)₃—OPO₃⁻—(CH₂)₂—N⁺(CH₃)₃, or —(CH₂)₄—C(O)—O—(CH₂)₂—OPO₃⁻—(CH₂)₂—N⁺(CH₃)₃; R⁵ and R⁶ each is H; and R⁷ and R⁸ together with the carbon atoms to which they are attached form phenyl (3-(1,1,2-trimethyl-1H-benzo[e]indol-3-ium-3-yl)propyl phosphocholine, and 2-(2-(1,1,2-trimethyl-1H-benzo [e]indol-3-ium-3-yl)acetoxy)ethyl phosphocholine, respectively) (see Table 1).

As disclosed herein, by exposing an indolium-based monomer of the formula I, as defined in any one of the embodiments above, to a basic buffer, optionally together with an additional monomer selected from those listed above, a mixture of same (i.e., identical) or different oligomers is obtained. The term “oligomer” as used herein refers to a compound obtained in the presence of a basic buffer by either polymerization of said indolium-based monomer or a derivative thereof, e.g., an oxidized form of said indoulium-based monomer obtained in a process similar to which occurs in the oxidation of PDA in basic buffers, optionally together with said additional monomer or a derivative thereof, e.g., an oxidized form of said additional monomer obtained in a process similar to which occurs in the oxidation of PDA in basic buffers; or as a result of a different process such as an alkylation, which may be an alternative version of increase in molecular weight other than polymerization. The oligomers comprised within the composition of the present invention may be the same or different, i.e., may be different in size, e.g., in the number of the repeating units constituting each one of the oligomers (and consequently in molecular weight), as well as in the type of repeating units, i.e., their structures. In certain embodiments, the mixture of oligomers comprised within said composition is obtained by exposing an indolium-based monomer of the formula I only to a basic buffer. In other embodiments, the mixture of oligomers comprised within said composition is in fact a mixture of co-oligomers, obtained by exposing an indolium-based monomer of the formula I together with dopamine, L-dopa, norepinephrine, serotonin, or any mixture thereof (i.e., a mixture of dopamine and L-dopa; dopamine and norepinephrine; dopamine and serotonin; L-dopa and norepinephrine; L-dopa and serotonin; norepinephrine and serotonin; dopamine, L-dopa and norepinephrine; dopamine, L-dopa and serotonin; L-dopa, norepinephrine and serotonin; or dopamine, L-dopa, norepinephrine, and serotonin), to said buffer.

TABLE 1
Structures of specific compounds of the formula I disclosed herein
2,3,3-trimethyl-1-(4-sulfobutyl)indolium inner salt (In783)

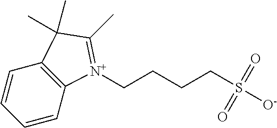 [SMILES: CC1=[N+](CCCCS(=O)(=O)[O-])c2ccccc2C1(C)C]

1,1,2-trimethyl-3-(4-sulfobutyl)benz[e]indolium inner salt (In820)

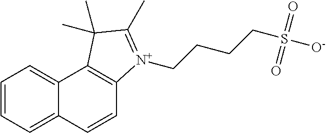 [SMILES: CC1=[N+](CCCCS(=O)(=O)[O-])c2ccc3ccccc3c2C1(C)C]

3-(1,1,2-trimethyl-1H-benzo[e]indol-3-ium-3-yl)propyl
phosphocholine

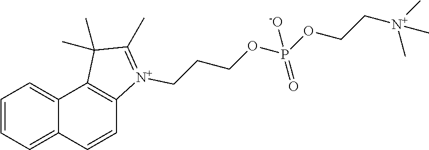

2-(2-(1,1,2-trimethyl-1H-benzo[e]indol-3-ium-3-yl)acetoxy)ethyl
phosphocholine

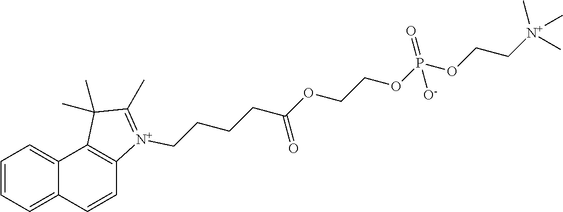 [SMILES: CC1=[N+](CCCCC(=O)OCCOP(=O)([O-])OCC[N+](C)(C)C)c2ccc3ccccc3c2C1(C)C]

Suitable basic buffers for use according to the present invention include, without being limited to, tris(hydroxymethyl)aminomethane (TRIS) buffer, 4-(2-hydroxyethyl)-1-piperazineethanesulfonic acid (HEPES) buffer, 3-[N-tris(hydroxymethyl)methylamino]-2-hydroxypropanesulfonic acid (TAPSO) buffer, N-[tris(hydroxymethyl)methyl]glycine (Tricine) buffer, 2-(bis(2-hydroxyethyl)amino)acetic acid (Bicine) buffer, [tris(hydroxymethyl)methylamino]propanesulfonic acid (TAPS) buffer, a borate-based buffer such as sodium borate, a tetraborate-based buffer such as sodium tetraborate and disodium tetraborate, or a bicarbonate-based buffer such as sodium bicarbonate buffer, potassium bicarbonate buffer, ammonium bicarbonate buffer, triethylammonium bicarbonate buffer, and carbonate bicarbonate buffer. In particular embodiments, said basic buffer is a bicarbonate-based buffer, such as sodium bicarbonate buffer, preferably 0.1M sodium bicarbonate buffer.

In certain embodiments, the mixture of oligomers comprised within the composition of the invention has been obtained by exposing the indolium-based monomer of formula I as defined in any one of the embodiments above, optionally together with an additional monomer as defined above or a combination thereof, to said basic buffer at a temperature of from about room temperature, i.e., 18-25° C., to about 150° C., e.g., from about 20° C. to about 120° C., from about 30° C. to about 115° C., from about 40° C. to about 110° C., from about 50° C. to about 105° C., or from about 60° C., 70° C. or 80° C. to about 100° C., preferably about 90° C.

In certain embodiments, the indolium-based monomer of the formula I as defined in any one of the embodiments above is exposed to said basic buffer for at least 2 hours, e.g., for about 4, 6, 8, 10, 12, 14, 16, 18, 20, 22, 24, 26, 28, 30, 32, 24, or 36 hours.

In certain embodiments, the concentration of the indolium-based monomer of the formula I as defined in any one of the embodiments above in said basic buffer, i.e., when exposed to said basic buffer (prior to oligomerization), is from about 1 mg/ml to about 100 mg/ml, e.g., from about 2 mg/ml to about 50 mg/ml, from about 4 mg/ml to about 40 mg/ml, from about 6 mg/ml to about 30 mg/ml, from about 8 mg/ml to about 20 mg/ml, or from about 2 mg/ml to about 10 mg/ml.

In certain embodiments, the mixture of oligomers comprised within the composition of the invention has been obtained by exposing an indolium-based monomer of the formula I as defined in any one of the embodiments above, optionally together with an additional monomer as defined above or a combination thereof, to a bicarbonate-based buffer such as sodium bicarbonate (e.g., 0.1M sodium bicarbonate), at a temperature of from about 50° C., 60° C., 70° C., or 80° C. to about 150° C., e.g., about 90° C., and for at least 2 hours, e.g., for about 4, 6, 8, 10, 12, 14, 16, 18, 20, 22, 24, 26, 28, 30, 32, 24, or 36 hours, wherein the concentration of said indolium-based monomer in said buffer, prior to oligomerization, is from about 2 mg/ml to about 50 mg/ml, from about 4 mg/ml to about 40 mg/ml, from about 6 mg/ml to about 30 mg/ml, from about 8 mg/ml to about 20 mg/ml, or from about 2 mg/ml to about 10 mg/ml, e.g., about 5 mg/ml.

In other embodiments, the mixture of oligomers comprised within the composition of the invention has been obtained by exposing an indolium-based monomer of the formula I as defined in any one of the embodiments above, optionally together with an additional monomer as defined above or a combination thereof, to a bicarbonate-based buffer such as sodium bicarbonate (e.g., 0.1M sodium bicarbonate), at room temperature, i.e., a temperature within the range of 18-25° C., for at least 12 hours, e.g., for about 14, 16, 18, 20, 22, 24, 26, 28, 30, 32, 24, or 36 hours, wherein the concentration of said indolium-based monomer in said buffer, prior to oligomerization, is from about 2 mg/ml to about 50 mg/ml, from about 4 mg/ml to about 40 mg/ml, from about 6 mg/ml to about 30 mg/ml, from about 8 mg/ml to about 20 mg/ml, or from about 2 mg/ml to about 10 mg/ml, e.g., about 5 mg/ml. In particular such embodiments, the product (i.e., oligomers-containing composition) thus obtained is further incubated at room temperature for at least 24 hours and up to several days, e.g., for about 24 hours to about 72 hours, for about 36 hours to about 60 hours, or for about 48 hours to about 54 hours, a period referred to herein as “aging period”.

In certain embodiments, the mixture of oligomers comprised within the composition of the invention has been obtained by exposing 1,1,2-trimethyl-3-(4-sulfobutyl)benz[e]indolium inner salt (In820) to a basic buffer, e.g., by polymerization of said indolium-based monomer in the presence of said basic buffer. In particular such embodiments, said basic buffer is a bicarbonate-based buffer such as sodium bicarbonate buffer, e.g., 0.1M sodium bicarbonate buffer. Such an oligomer mixture may be obtained by exposing In820 to said basic buffer at a temperature ranging from room temperature to about 150° C. (e.g., from about 20° C. to about 120° C., from about 30° C. to about 115° C., from about 40° C. to about 110° C., from about 50° C. to about 105° C., or from about 60° C., 70° C. or 80° C. to about 100° C.), for at least 2 hours (e.g., for about 4, 6, 8, 10, 12, 14, 16, 18, 20, 22, 24, 26, 28, 30, 32, 24, or 36 hours), wherein the concentration of the In820 in said basic buffer, prior to oligomerization, is from about 1 mg/ml to about 100 mg/ml (e.g., from about 2 mg/ml to about 50 mg/ml, from about 4 mg/ml to about 40 mg/ml, from about 6 mg/ml to about 30 mg/ml, from about 8 mg/ml to about 20 mg/ml, or from about 2 mg/ml to about 10 mg/ml).

In other embodiments, the mixture of oligomers comprised within the composition of the invention comprises co-oligomers, obtained by exposing 1,1,2-trimethyl-3-(4-sulfobutyl)benz[e]indolium inner salt (In820), together with an additional monomer selected from dopamine, L-dopa, norepinephrine, serotonin, and a combination thereof, to a basic buffer, e.g., by polymerization of said indolium-based monomer with said additional monomer in the presence of said basic buffer. In particular such embodiments, said basic buffer is a bicarbonate-based buffer such as sodium bicarbonate buffer, e.g., 0.1M sodium bicarbonate buffer. In certain more particular such embodiments, said mixture of co-oligomers is obtained wherein said additional monomer is dopamine, L-dopa, or a combination thereof, e.g., wherein said additional monomer is a mixture of dopamine and L-dopa, and the molar ratio between the In820, said dopamine, and said L-dopa is about 1:1:1, respectively. In other more particular such embodiments, said mixture of co-oligomers is obtained wherein said additional monomer is dopamine or serotonin, e.g., wherein the molar ratio between the In820 and said dopamine or serotonin is about 2:1, respectively. Such a co-oligomer mixture may be obtained by exposing In820 together with said additional monomer to said basic buffer at a temperature ranging from room temperature to about 150° C. (e.g., from about 20° C. to about 120° C., from about 30° C. to about 115° C., from about 40° C. to about 110° C., from about 50° C. to about 105° C., or from about 60° C., 70° C. or 80° C. to about 100° C.), for at least 2 hours (e.g., for about 4, 6, 8, 10, 12, 14, 16, 18, 20, 22, 24, 26, 28, 30, 32, 24, or 36 hours), wherein the concentration of the In820 in said basic buffer, prior to oligomerization, is from about 1 mg/ml to about 100 mg/ml (e.g., from about 2 mg/ml to about 50 mg/ml, from about 4 mg/ml to about 40 mg/ml, from about 6 mg/ml to about 30 mg/ml, from about 8 mg/ml to about 20 mg/ml, or from about 2 mg/ml to about 10 mg/ml).

The most frequent causes for low oral bioavailability of a drug are attributed to poor water solubility and low permeability. All drugs have been divided into four classes: class I refers to high soluble and high permeable drugs, class II refers to low soluble and high permeable drugs, class III refers to high soluble and low permeable, and class IV refers to low soluble and low permeable drugs.

Water solubility also plays a major role for other dosage forms like parenteral formulations, and it is one of the important parameters to achieve desired concentration of drug in systemic circulation for achieving required pharmacological response. Poorly water-soluble drugs often require high doses in order to reach therapeutic plasma concentrations after oral administration. Low aqueous solubility is the major problem encountered with formulation development of new chemical entities as well as generic development, as any drug to be absorbed must be present in the form of an aqueous solution at the site of absorption.

As shown in the Experimental section herein, the mixture of oligomers disclosed herein is useful in stabilizing a hydrophobic substance such as a drug in an aqueous medium, by forming stable nanoparticles comprising said mixture of oligomers and said hydrophobic substance, e.g., by encapsulating said hydrophobic substance by said oligomers.

In a particular such aspect, the present invention thus provides a hydrophobic substance-containing composition, i.e., a composition comprising a mixture of oligomers according to any one of the embodiments above, which further comprises a hydrophobic substance, e.g., a hydrophobic drug or dietary supplement, that is stabilized by said oligomers.

In certain embodiments, the hydrophobic substance-containing composition disclosed herein comprises a mixture of oligomers obtained by exposing 1,1,2-trimethyl-3-(4-sulfobutyl)benz[e]indolium inner salt (In820) to a basic buffer, e.g., a bicarbonate-based buffer such as sodium bicarbonate buffer, preferably 0.1M sodium bicarbonate buffer.

In other embodiments, the hydrophobic substance-containing composition disclosed herein comprises a mixture of co-oligomers obtained by exposing 1,1,2-trimethyl-3-(4-sulfobutyl)benz[e]indolium inner salt (In820), together with an additional monomer selected from dopamine, L-dopa, norepinephrine, serotonin, and a combination thereof, to a basic buffer, e.g., a bicarbonate-based buffer such as sodium bicarbonate buffer, preferably 0.1M sodium bicarbonate buffer. In certain particular such embodiments, said mixture of co-oligomers is obtained wherein said additional monomer is dopamine, L-dopa, or a combination thereof, e.g., wherein said additional monomer is a mixture of dopamine and L-dopa, and the molar ratio between the In820, said dopamine, and said L-dopa is about 1:1:1, respectively. In other particular such embodiments, said mixture of co-oligomers is obtained wherein said additional monomer is serotonin or dopamine, e.g., wherein the molar ratio between the In820 and said serotonin or dopamine is about 2:1, respectively.

The hydrophobic substance-containing composition disclosed herein may be, e.g., a pharmaceutical- or nutraceutical composition, optionally further comprising a pharmaceutically- or nutraceutically acceptable carrier and/or excipient, respectively.

The term “hydrophobic drug” as used herein refers to a chemical substance which is insoluble or poorly soluble in an aqueous medium, and when administered to a subject, produces a biological (e.g., therapeutical) effect. More specifically, it is used to, e.g., treat, cure, or prevent a disease. The term “hydrophobic dietary supplement” as used herein refers to a chemical substance that is insoluble or poorly soluble in an aqueous medium, which when administered to a subject, is useful as a dietary/nutritional supplement, i.e., may help improving or maintaining the overall health of said subject or meeting its daily requirements of essential nutrients.

The term “pharmaceutically acceptable carrier/excipient” as used herein refers to a non-active ingredient such as a solvent, dispersion medium, preservative, antioxidant, coating, isotonic and absorption delaying agent, and the like, that is compatible with pharmaceutical administration, and does not produce an adverse, allergic, or other untoward reaction when administered to a mammal or human as appropriate. For human administration, compositions should meet sterility, pyrogenicity, and general safety and purity standards as required by, e.g., the U.S. Food and Drug Administration (FDA), or the European Medicines Agency (EMA).

The term “nutraceutically acceptable carrier/excipient” as used herein refers to a non-active ingredient as defined above, that is compatible with nutraceutical administration, and does not produce an adverse, allergic, or other untoward reaction when administered to a mammal or human as appropriate.

In certain embodiments, the hydrophobic substance-containing composition of the invention is in the form of a suspension of nanoparticles in an aqueous liquid such as water and a buffer, wherein said nanoparticles each comprising said mixture of oligomers (or co-oligomers) and said hydrophobic drug. The size of such nanoparticles may range from about 20 nm to about 400 nm, e.g., from about 30 nm to about 300 nm, from about 40 nm to about 200, from about 50 nm to about 150 nm, from about 50 nm to about 100 nm, from about 60 nm to about 90 nm, or from about 70 nm to about 80 nm.

The term “suspension” as used herein refers to a heterogeneous mixture of a fluid, i.e., and aqueous medium such as water and buffer, that contains solid particles in which the solute particles do not dissolve, but get suspended throughout the bulk of the fluid.

In other certain embodiments, the hydrophobic substance-containing composition of the invention is in the form of a powder.

In certain embodiments, the hydrophobic substance comprised within the hydrophobic substance-containing composition of the invention, according to any one of the embodiments above, is a hydrophobic drug. Such a hydrophobic drug may be, without being limited to, an anticancer (chemotherapeutic) drug, antibacterial drug, antibiotic drug, antiseptic drug, antiviral drug, antifungal drug, immunosuppressive drug, anti-hyperlipidemic drug, nonsteroidal anti-inflammatory drug, cardiac drug, anticoagulant drug, diuretic drug, antiemetic drug, antihistamine drug, neurological drug, psychoactive drug, narcotic drug, bioactive peptide, steroid, hormone such as a peptide hormone, steroid hormone and thyroid hormone, hormone replacement therapy, selective estrogen receptor modulator (SERM), 5-alpha reductase inhibitor, interferon, interleukin, drug of abuse, alkaloid, nucleic acid, pesticide, prostaglandin, or vitamin.

Non-limiting examples of anticancer drugs include protein kinase inhibitors such as nilotinib, sorafenib, staurosporine, midostaurin, ibrutinib, trametinib, regorafenib, ponatinib, afatinib, pazopanib, rociletinib, dasatinib, ceritinib, ulixertinib, cabozantinib, nintedanib, selumetinib, thiazovivin, osimertinib, defactinib, idelalisib, taselisib, mubritinib, infigratinib, duvelisib, lapatinib, and avapritinib; angiogenesis and myeloma cell growth inhibitor such as pomalidomide; an antiandrogen medication such as bicalutamide; an anthracycline antibiotic such as valrubicin; a B-cell lymphoma 2 inhibitor such as navitoclax, a taxane-based drug such as paclitaxel and docetaxel; an antitumor antibiotic such as tanespimycin; a benzamide histone deacetylase inhibitor such as mocetinostat; a cancer cell stemness inhibitor such as napabucasin; a flavagline such as rocaglamide; a Hedgehog signaling pathway targeting agent such as vismodegib; a transforming growth factor beta (TGF-β) inhibitor such as galunisertib; a poly ADP ribose polymerase (PARP) inhibitor such as talazoparib; a retinoid-based drug such as alitretinoin; a topoisomerase inhibitor such as camptothecin, etoposide, and irinotecan; an epidermal growth factor receptor (EGFR) inhibitor such as gefitinib and erlotinib; a mammalian target of rapamycin (mTOR) inhibitor such as everolimus and rapamycin; a proteasome inhibitor such as carfilzomib; a nonsteroidal antiandrogen (NSAA) medication such as enzalutamide; non-limiting examples of antibacterial drugs include soluble adenylyl cyclase inhibitors such as bithionol; non-limiting examples of anti-hyperlipidemic drugs include probucol; non-limiting examples of 5-alpha reductase inhibitors include dutasteride; non-limiting examples of nonsteroidal anti-inflammatory drugs include cyclooxygenase-2 inhibitors such as celecoxib; non-limiting examples of selective estrogen receptor modulators include ospemifene; and non-limiting examples of immunosuppressive drugs include calcineurin inhibitors such as tacrolimus and cyclosporine.

In other certain embodiments, the hydrophobic substance comprised within the hydrophobic substance-containing composition of the invention, according to any one of the embodiments above, is a dietary supplement such as cholecalciferol and curcumin.

In further certain embodiments, the hydrophobic substance comprised within the hydrophobic substance-containing composition of the invention, according to any one of the embodiments above, is a hydrophobic drug comprising a 2-(N-anilino)pyrimidine group coupled to π-conjugated system directly or via a nitrogen atom. In particular such embodiments, said hydrophobic drug is a protein kinase inhibitor exhibiting an aggregation-induced emission (AIE), i.e., a brightened emission in the aggregated form.

The compositions provided by the present invention may be prepared by conventional techniques known in the art, e.g., as described in Remington: The Science and Practice of Pharmacy, 19^{th} Ed., 1995. In particular, the compositions may be prepared, e.g., by uniformly and intimately bringing the oligomers/co-oligomers-containing composition disclosed herein when further comprising said hydrophobic drug (altogether referred to herein as “the active agent”) into association with a liquid carrier, a finely divided solid carrier, or both, and then, if necessary, shaping the product into the desired formulation.

Hydrophobic substance-containing pharmaceutical compositions according to the present invention may be formulated for enteral, e.g., oral, sublingual, supra-lingual, buccal, or rectal, administration; parenteral, e.g., intravenous, intraarterial, intrathecal, intrapleural, intratracheal, intraperitoneal, intramuscular, intranasal, transdermal, subcutaneous, vaginal, topical, nasal, or ophthalmic (e.g., as eye drops), administration; or for inhalation. Hydrophobic substance-containing nutraceutical compositions according to the invention may be formulated for oral, sublingual, supra-lingual, or buccal administration.

Pharmaceutical compositions formulated for oral administration may be in the form of a liquid, e.g., a solution in an edible solvent such as ethanol, tincture, syrup, or elixir; a semi-solid; or a solid such as tablets, caplets, pills, troches, lozenges, dispersible powder or granules, hard or soft capsules, and sachets. In certain embodiments, the pharmaceutical composition is in the form of a bi- or multilayer tablet, in which each one of the layers comprise the active agent, and the layers are optionally separated by an intermediate, inactive layer, e.g., a layer comprising one or more disintegrants.

Useful dosage forms of the pharmaceutical compositions include orally disintegrating systems including, but not limited to, solid, semi-solid and liquid systems including disintegrating or dissolving tablets, soft or hard capsules, gels, fast dispersing dosage forms, controlled dispersing dosage forms, caplets, films, wafers, ovules, granules, buccal/mucoadhesive patches, powders, freeze dried (lyophilized) wafers, chewable tablets which disintegrate with saliva in the buccal/mouth cavity and combinations thereof. Useful films include, but are not limited to, single layer stand-alone films and dry multiple layer stand-alone films.

In certain embodiments, the pharmaceutical compositions are formulated for oral administration, and are in the form of matrix tablets wherein the release of the active agent is controlled by having said active agent diffuse through a gel formed after the swelling of a hydrophilic polymer brought into contact with dissolving liquid (in vitro) or gastro-intestinal fluid (in vivo). Many polymers have been described as capable of forming such gel, e.g., derivatives of cellulose, in particular the cellulose ethers such as hydroxypropyl cellulose, hydroxymethyl cellulose, methylcellulose or methyl hydroxypropyl cellulose, and among the different commercial grades of these ethers are those showing fairly high viscosity. In other embodiments, the tablets are formulated as bi- or multi-layer tablets, made up of two or more distinct layers of granulation compressed together with the individual layers lying one on top of another, with each separate layer containing a different active agent (i.e., a different hydrophobic drug stabilized by an oligomers/co-oligomers mixture as disclosed herein). Bilayer tablets have the appearance of a sandwich since the edge of each layer or zone is exposed.

Pharmaceutical compositions for oral administration might be formulated so as to inhibit the release of the active agent in the stomach, i.e., delay the release of the hydrophobic drug until at least a portion of the dosage form has traversed the stomach, in order to avoid the acidity of the gastric contents from hydrolyzing said hydrophobic drug. Particular such compositions are those wherein the active agent is coated by a pH-dependent enteric-coating polymer. Examples of pH-dependent enteric-coating polymer include, without being limited to, Eudragit® S (poly(methacrylicacid, methylmethacrylate), 1:2), Eudragit® L 55 (poly (methacrylicacid, ethylacrylate), 1:1), Kollicoat® (poly(methacrylicacid, ethylacrylate), 1:1), hydroxypropyl methylcellulose phthalate (HPMCP), alginates, carboxymethylcellulose, and combinations thereof. The pH-dependent enteric-coating polymer may be present in the composition in an amount from about 10% to about 95% by weight of the entire composition.

Another contemplated formulation is depot systems, based on biodegradable polymers. As the polymer degrades, the active agent is slowly released. The most common class of biodegradable polymers is the hydrolytically labile polyesters prepared from lactic acid, glycolic acid, or combinations of these two molecules. Polymers prepared from these individual monomers include poly (D,L-lactide) (PLA), poly (glycolide) (PGA), and the copolymer poly (D,L-lactide-co-glycolide) (PLG).

Pharmaceutical compositions for oral administration may further comprise one or more agents selected from sweetening agents, flavoring agents, coloring agents and preserving agents in order to provide pharmaceutically elegant and palatable preparations. In addition, said compositions may comprise one or more pharmaceutically acceptable excipients. For example, a tablet may comprise at least one filler, e.g., lactose, ethylcellulose, microcrystalline cellulose, silicified microcrystalline cellulose; at least one disintegrant, e.g., cross-linked polyvinylpyrrolidinone; at least one binder, e.g., polyvinylpyridone, hydroxypropylmethyl cellulose; at least one surfactant, e.g., sodium laurylsulfate; at least one glidant, e.g., colloidal silicon dioxide; and at least one lubricant, e.g., magnesium stearate.

Pharmaceutical compositions formulated for parenteral administration, i.e., for administration elsewhere in the body than the mouth and alimentary canal, may be in the form of a sterile, optionally injectable, aqueous or oleaginous suspension, which may be formulated according to the known art using suitable dispersing, wetting or suspending agents. The sterile injectable preparation may also be an injectable solution or suspension in a non-toxic parenterally acceptable diluent or solvent. Acceptable vehicles and solvents that may be employed include, without limiting, water, Ringer's solution, polyethylene glycol (PEG), 2-hydroxypropyl-β-cyclodextrin (HPCD), a surfactant such as Tween-80, and isotonic sodium chloride solution.

In another aspect, the present invention relates to a method for identifying whether a compound or mixture of compounds is capable of stabilizing (both in storage and in vivo) a hydrophobic substance such as a hydrophobic-drug and dietary supplement, said hydrophobic substance exhibiting a fluorescence emission in the aggregated state that is significantly increased compared to its fluorescence emission in the non-aggregated, i.e., soluble, state, said method comprising the steps of:
(i) mixing an aqueous solution of said compound or mixture of compounds with said hydrophobic substance to obtain a suspension of nanoparticles each comprising said compound or mixture of compounds and said hydrophobic substance;
(ii) imaging the fluorescence emission of the suspension obtained; and
(iii) comparing said fluorescence emission with the fluorescence emission of said hydrophobic substance in its aggregated state optionally divided by white light (brightfield),
wherein a significant decrease, e.g., a decrease of at least about 20%, 30%, 40%, 50%, 60%, 70%, 80%, or more, in the fluorescence emission of said substance in said suspension compared to its fluorescence emission in the aggregated state optionally divided by white light (brightfield) indicates that said compound or mixture of compounds is capable of stabilizing said substance.

In certain embodiments, the compounds or mixture of compounds utilized according to the method of the invention is a mixture of oligomers or co-oligomers obtained by exposing an indolium-based monomer of the formula I, optionally with an additional monomer selected from dopamine, L-dopa, norepinephrine, serotonin, and a mixture thereof, to a basic buffer, according to any one of the embodiments above.

In certain embodiments, the hydrophobic substance mixed in step (i) of the method of the invention, according to any one of the embodiments above, is a hydrophobic drug or dietary supplement, each as defined hereinabove.

Unless otherwise indicated, all numbers expressing, e.g., amounts/concentrations of components, temperatures, sizes, and ratios between components, used in this specification, are to be understood as being modified in all instances by the term “about”. Accordingly, unless indicated to the contrary, the numerical parameters set forth in this specification are approximations that may vary by up to plus or minus 10% depending upon the desired properties to be obtained by the present invention.

The invention will now be illustrated by the following non-limiting Examples.

### EXAMPLES

### Study 1. Automated Discovery of Nanomaterials Via Drug Aggregation Induced Emission (AIE)

### Materials and Methods

Materials and reagents. All non-drug chemicals were purchased from Sigma Aldrich (St. Louis, MO). Dimethylsulfoxide (DMSO) was purchased from Carlo Erba (Emmendingen, Germany). Drugs were purchased from LC-Laboratories (Woburn, MA) and MedChemExpress.com.

Extractive literature search for AIEgenic drugs. We used the SPIKE over PubMed abstracts engine to identify published drugs with AIE activity. SPIKE is a natural language processing based search engine, which can extract entities that are in co-occurrence with each other: https://spike.apps.allenai.org/datasets/pubmed. The queries used for this task were basic Boolean search:
:w={FDA_DRUGS_2500} (in house list of FDA approved drugs from DrugBank.com) with a document abstract filters: “aggregation-induced emission”|“aggregation induced emission”|“AIE”. A detailed tutorial on how to use SPIKE for extractive search may be found in https://spike.apps.allenai.org/datasets.

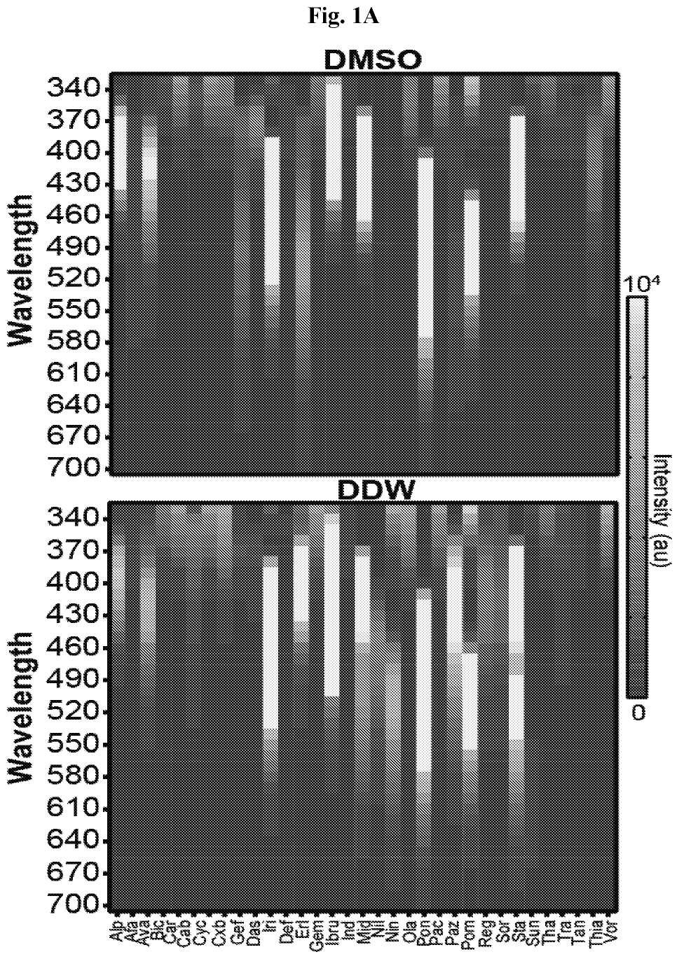

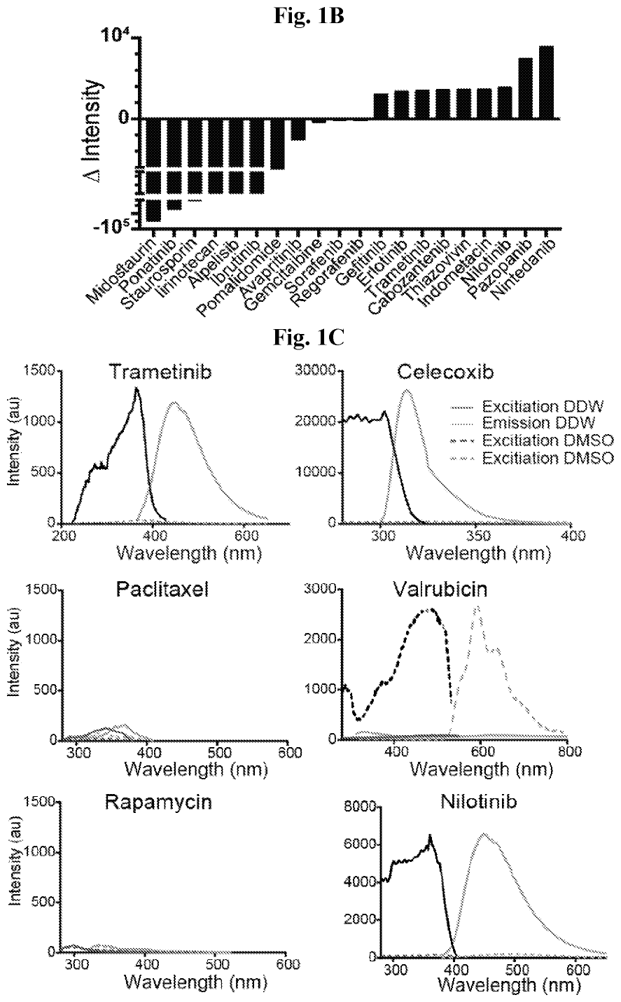

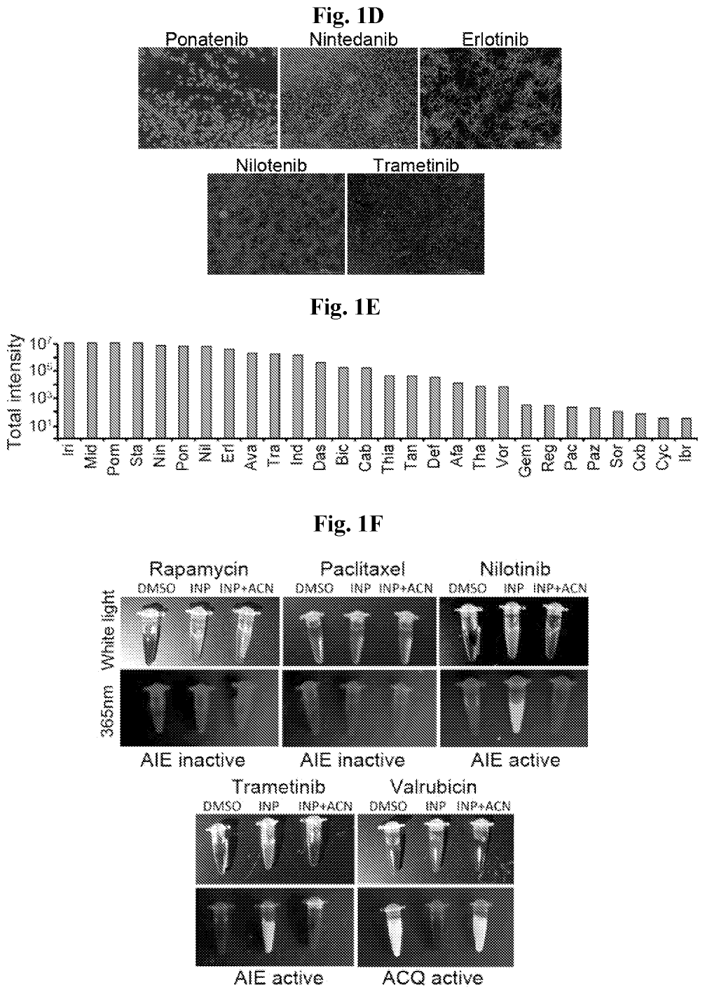

Screening for AIEgenic drugs. 30 different hydrophobic drugs were dissolved in DMSO at 1 mg/ml concentration, and 20 μl of each drug was added to a 96 well plate containing 180 μl of either double distilled water (DDW) or DMSO in each well. The fluorescence spectrum was evaluated with Synergy N1 (BioTek®) plate reader with 320 nm excitation and emission from 330 to 700 nm in 10 nm steps. The data was analyzed with Prism GraphPad® 9, where DDW readouts were subtracted from the DMSO readouts to generate the waterfall plot in FIG. 1B.

Imaging fluorescent drug aggregates with automated microscopy. To image fluorescent drug aggregates, we used the same concentrations of the drugs in the screening experiments in 96 wells. The wells were imaged in the 4′,6-diamidino-2-phenylindole (DAPI) channel (Ex. 377 nm, Em. 447 nm) of LionHeart (BioTek®) with a 10 ms exposure, 10% digital gain, 100% LED intensity and image based autofocus. To quantify the AIE effect, two image analysis protocols were used from the supplier image analysis software, Gen5+ Data Analysis. The first analysis was image statistics—total intensity (FIG. 1E) and in the following experiments—cellular analysis: object sum area. The first method is aimed to quantify the yield of fluorescence, whereas the second method is aimed to characterize the aggregate properties such as size and number. The second method was used for product selection in the automated nano-formulation process. It is important to note that the DAPI channel with 377 nm is the optimal setup. We have tried to image drug aggregates in 405 nm Ex with inferior quality images.

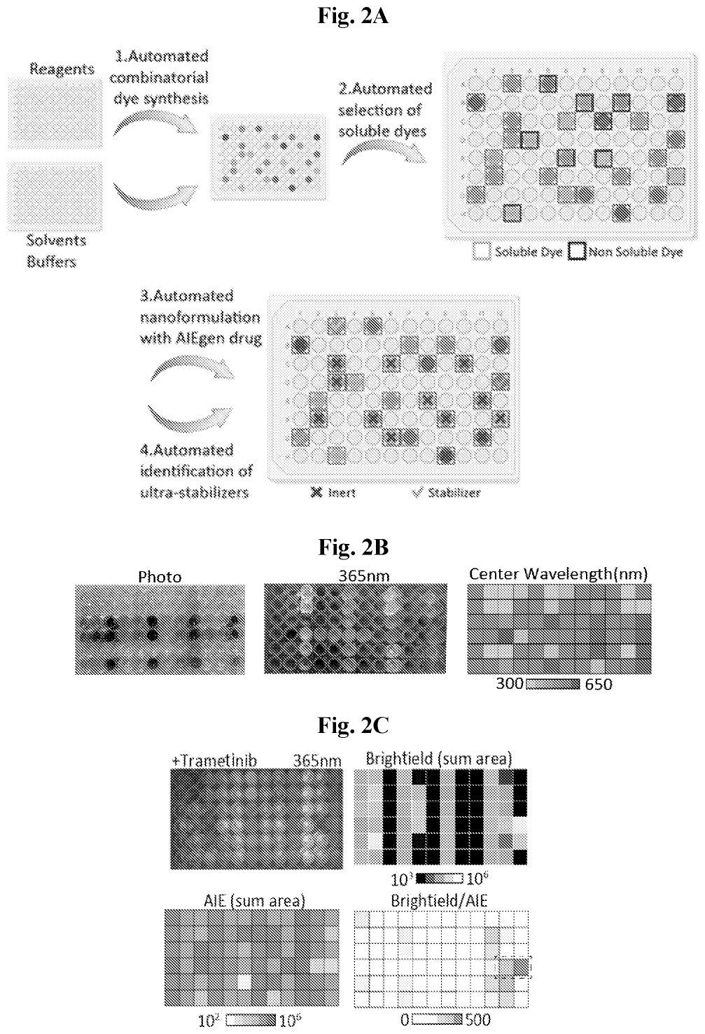

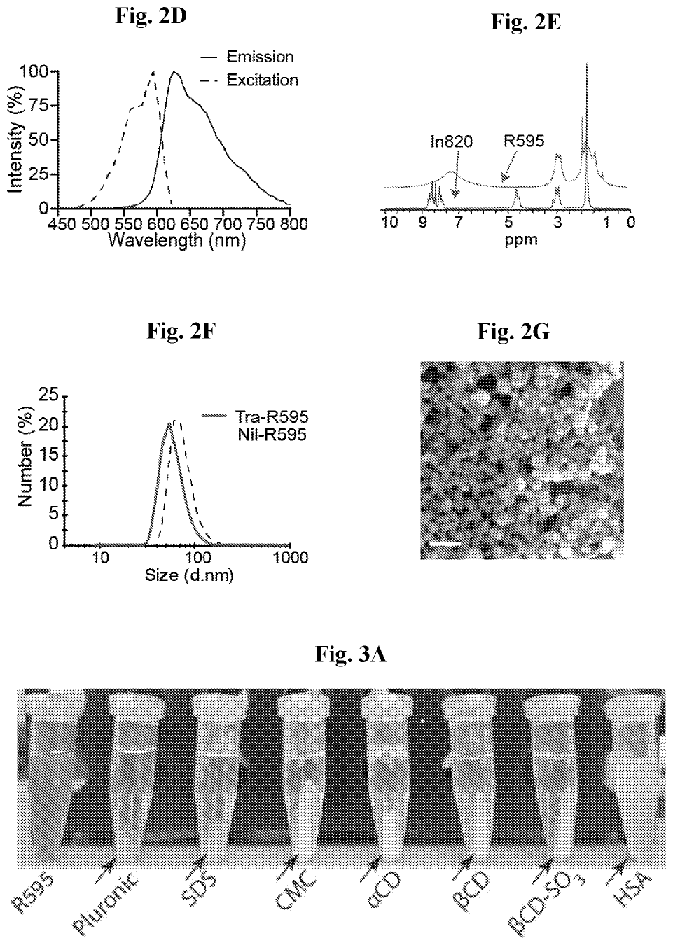

Automated combinatorial dye synthesis. The Andrew Alliance+ electronic pipetting robot (Waters*, USA) was used for both synthesis and nano-formulation. The design of reagents arrangement and synthesis sequences are shown in FIG. 2. For dye synthesis, solvents which are compatible with polypropylene tubes were used: DMSO, 0.1M NaHCO₃, DDW, EtOH, 1 or 0.01 mM NaOH, and 5 mM tris(hydroxymethyl)aminomethane. The bis(aldehydes) that were used were benzene 1,3,5 tricabocaldehyde, isophtaldehyde, and paraformaldehyde; all 10 mg/ml dissolved in DMSO. The indoliums used were (2,3,3-trimethyl-1-(4-sulfobutyl)indolium (In783) and 1,1,2-trimethyl-3-(4-sulfobutyl) benzndolium (In820), both 10 mg/ml dissolved in DDW, as well as 1,2,3,3-tetramethyl-3H-indol-1-ium (non-sulfated form of In783), 10 mg/ml dissolved in DMSO. Briefly, a combination matrix of 72 different conditions was performed in both 24 and 96 well plates with no apparent difference between them. The digital protocol can be accessed freely from the Andrew Alliance website. The final volume in each well was set for 200 μl.

Automated nanoformulation. For nanoformulation, a 10 mg/ml AIEgenic drug (trametinib or nilotinib) in DMSO stock solutions were used and a simple addition protocol was written to add 20 μl of drug into 100 μl mixture. The plates were imaged with Lionheart as described above, using the cellular analysis method (Gen5+ Software).

Optimized preparation protocol for R595 and R555 from the combinatorial screen. In a 15 ml polypropylene conical tube, 50 mg of 2,3,3-trimethyl-1-(4-sulfobutyl) indolium or 1,1,2-trimethyl-3-(4-sulfobutyl)benz[e]indolium inner salt (Sigma/Merck) were dissolved in 5 ml of 0.1M sodium bicarbonate buffer for R555 and R595 synthesis, respectively. The mixture was heated to 90° C. and shaken for 4 h in Eppendorf ThermoMixer®, and then remained at room temperature for another 48 h before used for nanoparticles preparations.

Preparation of additional derivatives. In a 1.5 ml polypropylene conical tube, 5 mg of 2,3,3-trimethyl-1-(4-hydroxypropyl) indolium or 1,1,2-trimethyl-3-(4-carboxybutyl)benz [e]indolium inner salt were dissolved in 0.5 ml of 0.1M sodium bicarbonate buffer. The mixture was heated to 90° C. and shaken for 4 h in Eppendorf ThermoMixer®, and then remained at room temperature for another 48 h before used for nanoparticles preparations.

Manual preparation of nanoparticles. 100 μL of drugs dissolved in DMSO (10 mg/ml) were added under slight vortexing to 0.5 ml of 0.1 M sodium bicarbonate buffer containing 1.6 mg/ml R595 (100 μl from 10 mg/ml stock solution in 0.5 ml). For comparison with other stabilizers, the R595 was replaced with the following excipients: Pluronic F-127, sodium lauryl sulfate (SDS), carboxymethyl cellulose (CMC), α-cyclodextrin (αCD), β-cyclodextrin (βCD), βCD-SO₃, and human serum albumin (HSA). All compounds were dissolved as 10 mg/ml in DDW. Further comparison of R595 with IR783/IR820 based nanoparticles was also performed in DDW. The solutions were centrifuged (30,000 g, 15 min, room temperature) and the pellet was resuspended in 1 ml of DDW. In the automation process, using the Andrew Alliance pipetting robot (Waters Corp, MA), the nanoparticles were purified using a PD-10 desalting column (Cytiva, Marlborough, MA) and 1.7 ml of eluent was collected after 2.5 ml void volume (defined by the vendor).

Characterization of nanoparticles. Dynamic light scattering (DLS) measurements were conducted using a Zetasizer Nano ZS (Malvern Panalytical, Malvern, UK).

Transmission electron microscope (TEM): nanoparticles specimens were prepared in a controlled environment vitrification system (CEVS) and loaded to a FEI Talos 200C High Resolution TEM (Thermo Fisher Scientific, Waltham, MA).

Scanning electron microscope (SEM): nanoparticles were loaded onto a silicon wafer and dehydrated for High Resolution SEM (Zeiss, Germany), operated by the Technion Center for Electron Microscopy of Soft Matter (Haifa, Israel) and the Technion EMC—The Electron Microscopy Center (Haifa, Israel) respectively.

Drug loading: nanoparticles diluted 1:10 in acetonitrile were measured with ultra-performance liquid chromatography (Acquity arc UHPLC, Waters Corp, MA) using a CORTECS C18 (4.6×50 mm 2.7 μm) column.

Cell lines. All cell lines were cultured in Dulbecco's Modified Eagle Medium (DMEM) supplemented with 10% fetal bovine serum, 2 mM L-glutamine, 100 units/ml penicillin, and 100 μg/ml streptomycin sulfate (all from Biological Industries, Israel) in a 37° C. and 5% CO₂ incubator. The KPL-KRAS^{G12D}P53^{-/-} lung cancer cell line was isolated and subculture from mice in the lab of Tyler Jacks, MIT, and the 3T3, B16F10 and EA.hy926 cell lines were provided by the Heller lab at MSKCC. The HCT116, Cal33-GFP and FaDu cell lines were a kind gift provided by the lab of Moshe Elkabets, Ben-Gurion University, Israel. The KPC cell line was kindly given by the Schroder lab.

Nanoparticles cellular uptake assay. Cells grown for 24 h on glass bottom 96-well plate (Eppendorf, Hamburg, Germany) were incubated for 3 h with 75 μg/ml of nanoparticles followed by 20 min with nuclear staining Hoescht (Invitrogen, Thermo Fisher Scientific, Waltham, MA). The cells were rinsed twice with 150 μl phosphate-buffered saline (PBS) and fixed with 4% paraformaldehyde for 30 min. Images were acquired using Lionheart FX Automated Microscope (BioTek, Agilent Technologies, Santa Clara, CA) equipped with 16-bit grayscale Sony CMOS camera and high-power LEDs. Identical exposure times and excitation intensities were applied for all cell types. Filter sets: nucleus: ex 377 nm, em 447 nm, R595 dye in nanoparticles: ex 530 nm, em 595 nm. Images were processed with either Gen5 3.09 (BioTek, Agilent Technologies, Santa Clara, CA) or ImageJ (NIH, Bethesda, Maryland) software.

Cell viability assay in 2D. Cells were seeded in 96 well plates at a 30% confluency and allowed to adhere for 24 h. All drugs were dissolved in DMSO. Nanoparticles were suspended in DDW and were added to the wells after dilution with growth medium. Untreated cells (control) were used to establish 100% viability. The effect on cell viability was measured both with Promega® CellTiter-Glo® (CTG) and with images taken by the Lionheart followed by a cell count feature provided by the vendor (BioTeK).

Cell viability in 3D (tumor spheroids). About 1000 cells of either HCT116 or KPL were seeded in round bottom ultra-low attachment plates (ULA, Corning, NY) and were incubated for 96 h for maturation. Drugs or nanoparticles were added in the appropriate dilution (in growth medium) and incubated for 72 h. The viability of the spheroids was quantified by Promega® CellTiter-Glo® 3D kit, according to the manufacture instruction. Spheroids size was monitored with Lionheart automated microscopy.

Animal studies. Mice were maintained and treated, and animal studies were conducted, according to protocols approved by the Institutional Animal Research Ethical Committee at the Technion—Israel Institute of Technology.

In vivo safety study. Animals were intraperitoneally injected with 1 mg/ml drug equivalent dose of nanoparticles, twice per week for 5 weeks; and viability and weight were measured every other day.

KRAS driven lung tumor studies. Six-weeks-old female C₅₇BL/6J mice strain were purchased from Envigo RMS. Inducing tumors in the lung was done by injecting 10⁵ KPL cells suspended in 200 μl of PBS intravenously (IV) via the tail vein. Mice were administered intraperitoneal (IP) either 10 mg/kg trametinib (free drug, FD) in DMSO or 10 mg/kg trametinib-R595 nanoparticles. For efficacy studies, treatments were administered biweekly for ten weeks. For safety studies, immunohistochemistry (H&E) was done on treated mice. Animals were euthanized using CO₂ inhalation. Mice were housed in air-filtered laminar flow cabinets with a 12-hr light/dark cycle and food and water ad libitum.

KRAS-PI3K driven colon cancer xenograft studies (HCT116). Six-week-old female Hsd:Athymic Nude-Foxn1nu mice, purchased from Envigo RMS, were injected with 5×10⁵ HCT116 human colorectal carcinoma cells subcutaneously in 100 μl culture media/Matrigel (Corning) at a 1:1 ratio. Animals were randomized into four groups, with n=8 tumors per group. Animals were treated with either trametinib (10 mg/kg, per os, PO) or trametinib-R595 nanoparticles purified by either PD10 or centrifuge (10 mg/kg, IP) twice a week. Tumor size was measured with a digital caliper, and tumor volume was calculated using the formula: (length×width²)×(π/6). Animals were euthanized using CO₂ inhalation. Mice were housed in air-filtered laminar flow cabinets with a 12 h light/dark cycle and food and water ad libitum. For targeting and biodistribution experiments, mice were injected with trametinib-R595 nanoparticles 2 weeks after tumor cells injection. Organs were harvested 24 h after injection and examined in an in vivo imaging system (IVIS).

In vivo imaging of nanoparticles with IVIS. 24 hours after IP injection of 200 μl (10 mg/ml) trametinib nanoparticles (purified by either PD10 or centrifuge), organs (liver, kidney, lungs, heart, spleen and tumor) were extracted and placed on a Petri dish. Images were taken with an IVIS imaging system (Xenogen Corp.). Radiance efficiency (photons sec⁻¹cm⁻²) was calculated for the tumor region of interest (ROI) using LivingImage V4.2 software.

Statistical analysis. Statistical analysis for in vitro and in vivo experiments was performed using GraphPad Prism (GraphPad 9 Software). A two-tailed Student's unpaired t-test was conducted to compare control vs. treated groups. The significance level was set at P<0.05. Independent experiments were conducted with a minimum of two biological replicates per condition to allow for statistical comparison. Error bars represent the standard error of the mean (s.e.m), and P values are indicated in the figure captions and main text. All cellular experiments were repeated at least two times. Survival plots of experimental metastasis models were analyzed using the Mantel-Cox log-rank test. For all in vivo experiments, the sample size was at least n=3 mice per treatment group. These sample sizes were chosen based on previous literature and our own expertise. Animal cohorts were randomly selected. Investigators were not blinded. Representative images of tissue histology were from at least five images of the same tissue, and duplicate staining was done for each tissue from experiments so that at least two out of five organs/tissues were embedded and stained.

Cryo-TEM. 10 μL from nanoparticles solution were applied to carbon-coated TEM grid, excess solution was removed by Whatman paper, and then plunged to a nitrogen-cooled liquid ethane. This rapid cooling (105° C.×s⁻¹) prevents the formation of ice crystals on the grid. The samples were kept in liquid nitrogen until imaging. Samples' imaging was carried under Talos (Thermo Fisher Scientific) TEM (Technion center for electron microscopy of soft matter) and analyzed by ImageJ software (National Institute of Health, USA).

HR-SEM imaging. 2 μL from nanoparticles solution were applied to a silicon wafer and placed in a desiccator under vacuum for 72 h for dehydration. High resolution scanning electron microscopy (HR-SEM) imaging was performed by the Technion Electron Microscopy Center on a Zeiss Ultra Plus high-resolution SEM, equipped with a Schottky field-emission gun. Specimens were imaged at acceleration voltages of 4 kV and a working distance of 3.7 mm. The Everhart Thornley (“SE2”) secondary electron imaging detector was used.

ATR-FTIR. Samples were lyophilized by Freeze zone 2.5 (Labconco, USA) at −54° C., 0.01 mbar for 48 hours. Transmittance was measured in Bruker's Fourier transform infrared (FT-IR) Alpha at 400-4000 cm⁻¹ to identify the chemical signature of the polymers. The measurements were done at the Chemical and Surface Analysis Lab—at The Schulich Faculty of Chemistry, the Technion.

MALDI-TOF. For MALDI (matrix-assisted laser desorption ionization)-TOF (time of flight) measurements the samples were sent to analysis at The Schulich Faculty of Chemistry, The Technion, after purification with size exclusion chromatography by PD-10 desalting column.

H-NMR. ¹H NMR (60 MHz, 320C) spectra of the monomers and their products were obtained in NMReady 60 Pro (GPE Scientific Ltd.). R595 and R555 were lyophilized by Freeze zone 2.5 (Labconco, USA) at −54° C., 0.01 mbar for 48 hr and then redissolved in D₂O, while monomers were dissolved directly in D₂O. Each sample was scanned for 760 min (10,000 scans) and data was analyzed by Mnova v.14 (Mestrelab research, Spain).

### Results and Discussion

Identification and characterization of strong AIEgenic-drugs. To streamline the automated process of stabilizing dye synthesis and validation, we first wanted to identify drugs with useful optical properties such as AIE and ACQ. Using the SPIKE search engine (Ravfogel et al., 2021; Taub-Tabib et al.), we performed an extractive literature search task on PubMed abstracts to identify drugs that were reported directly with the AIE phenomenon. Although we found 65 different drugs that were co-mentioned with AIE, only five drugs were reported to be AIEgens: lapatinib, phenothiazine, berberine, deferasirox, and rilpivirine. To expand the known space of AIEgenic drugs, we interrogated the fluorescence emission spectrum of 30 hydrophobic drugs in soluble state (DMSO) and compared it to their emission spectrum in the aggregated state (DDW) following excitation at 320 nm. Drugs that showed lower fluorescence intensity in the aggregated state (negative delta value) are defined as ACQ, while AIEgenic drugs are those exhibiting a positive subtraction value between florescence in DDW and DMSO. As shown in FIG. 1A, 10 drugs showed increased fluorescence in the solid state and labeled as AIEgens, 8 drugs had strong ACQ type of fluorescence, and the additional 12 had no significant optical activity. We calculated and sorted the differences between the maximum fluorescence intensities in DDW and DMSO, in order to cluster the drugs according to their fluorescence activity (FIG. 1B).

To further characterize this finding, we selected representative drugs, characterized their full emission and excitation spectrums, and observed that the emission center wavelength is highly variable between drugs (FIG. 1C). For example, nilotinib and trametinib had a center wavelength emission in the blue region of 450 nm, while other drugs like celecoxib had a center wavelength at the UV region of 350 nm. For comparison, we show drugs which had very little optical activity like rapamycin and paclitaxel.

To exploit AIE in an automated nano-formulation workflow, we wanted to characterize drug aggregates in an automated fluorescent microscope imaging. Images of the aggregated drugs aligned in a 96-well plate were acquired with a DAPI channel (ex.377, em. 447). Indeed, we were able to characterize both the AIE intensity together with different aggregate morphology in terms of shape and size (FIG. 1D). Interestingly, the ACQ drug ponatinib was fluorescent both in the soluble and in the aggregated states but was highly visible in the aggregated state with very low background noise (FIG. 1D). Another interesting phenomenon was observed with nintendanib which was fluorescent in the soluble state at 520 nm and highly fluorescent in the aggregated state at 447 nm with very low background noise. We observed that even though trametinib and nilotinib had a relatively low intensity compared to the background signal, they could still be easily viewed, processed, and ranked with image analysis (FIG. 1E).

Next, we wished to demonstrate that AIEgenic drugs can also be observed macroscopically with simple equipment such as UV lamp, and that this phenomenon is indeed dependent on the aggregation state, and it is reversible. FIG. 1F illustrates that aggregated drugs packed in IR783 nanoparticles displayed a very strong AIE activity, whereas this effect is canceled with the addition of acetonitrile which disintegrates the nanoparticles and dissolves the drug. The strongly fluorescent drug, valrubicin, having the opposite effect of ACQ, is shown as a control.

As we tried to generalize and understand this phenomenon in small molecule drugs, we looked at the chemical structures of the AIEgenic drugs and noted that they all had similar molecular motifs which is quite common in kinase inhibitors (Scheme 1). One recurring motif is comprised of a 2-(N-anilino)pyrimidine group coupled to another π-conjugated system directly or via a nitrogen atom, but not via an oxygen. Nitrogen seems to be important for both AIE and regular fluorescence. A surprising observation was made about thalidomide and its derivative pomalidomide. Pomalidomide, which differs from thalidomide only by one atom of nitrogen, was one of the most fluorescent drugs in our screen, whereas thalidomide had no fluorescence at all.

Automated synthesis and selection of stabilizers using AIEgenic drugs. After identifying several AIEgenic drugs, we thought to exploit their fluorescent aggregation readout as a product selection strategy for automated chemical synthesis of novel stabilizers. In this approach, we define the non-stable state of the formulation by its fluorescence intensity, rather than size. There are several advantages of using fluorescence instead of size which is commonly measured with DLS. First, standard DLS instruments are not high throughput as they work with cuvettes and are rarely equipped with autosamplers. Second, existing DLS with plate reading capabilities are expensive and difficult to handle as they are very sensitive to concentration, temperature and material type. These reasons favor the use of AIE in an automated system for nanoformulations. Our automated system is based on a liquid handling robot (Andrew Alliance) for reagents mixing and formulation coupled with an automated fluorescent microscope for product selection (Lionheart, BioTek). We selected the Andrew Alliance® robot as it is completely open, modular and can also perform size exclusion chromatography for purification in later stages (the complete process is illustrated in FIG. 2A). First, the liquid handling robot performs automated combinatorial synthesis using a library of reagents and solvents. Then, soluble dye products are recognized in images taken using automated microscopy. Next, the liquid handling robot formulates an AIEgeneic drug like trametinib or nilotinib with the dyes using nanoprecipitation. Finally, automated identification of the ultra-stabilizers is done using AIE fluorescence of non-stable formulations.

Since previously published stabilizers were based on indocyanine dyes (Shamay et al., 2018), we sought to design similar new molecules using a combinatorial synthetic screen with some of the original building blocks for IR783 and IR820 together with novel precursors.

We started with a small combinatorial screen of indoliums In783, non-sulfated In783 and In820, and 3 bis(aldehydes) which were expected to react without a catalyst (Narayanan and Patonay, 1995; Liu et al., 2014a). With the idea of using the dye products later in nanoparticles preparation in a one-pot synthesis without a purification step, we chose five “green” solvents which are completely compatible with the nanoprecipitation process: DMSO, sodium bicarbonate buffer, tris(hydroxymethyl)aminomethane (TRIS) buffer, ethanol, and sodium hydroxide. There were 72 different reaction mixtures of all possible reagents combinations, and as hypothesized, some would result in dye products with nanoparticles stabilizing properties.

After a 24 h incubation, the reaction mixtures yielded a variety of colorful products in the visible range (red, purple, yellow, and orange), some of which having strong fluorescent emission (FIG. 2B).

We characterized the absorbance spectrum of each product, and the center wavelengths ranging from 300 nm to 650 nm, were plotted (FIG. 2B, right panel). Adding to the products the AIEgenic drugs nilotinib and trametinib, which do not form stable nanoparticles with published stabilizers, we could see both drug and dye product fluorescence (FIG. 2C, left panel).

Images taken with the automated microscope for analyzing both the 377-nanometer channel together with the brightfield clearly show that in some cases there's a strong blue fluorescence indicating a significant drug aggregation while in other cases the blue is low and the brightfield is high, indicating low precipitation of both the dye and the drug (data not shown). The quantification of brightfield with AIE of the nanoparticles' reaction mixture showed that in most reactions there had been a strong AIE activity but in six of them, there is relatively low AIE fluorescence indicating low drug aggregation and high particle stability (FIG. 2C, three right panels). It is of note that some of the low AIE fluorescence can be accounted for the strong absorbance of the precipitate so that light cannot pass through the sample. Thus, the quantification and ranking of aggregation prevention is possible by dividing the AIE values by the brightfield values to identify the most potent stabilizers.

This analysis identified two potent dye products with absorbance peaks in 555 nm and 595 nm which we named R555 and R595 (FIG. 2C, right panel). To our surprise, the reaction mixtures for these dye products contained only indoliums reagents in bicarbonate buffer without aldehydes which are required for this type of dye synthesis. Despite searching the scientific literature and the SciFinder database, no publication reporting monomeric indolium reactions in weak basic buffers was found. In our experiment, we found that all three indoliums reacted in weak basic buffers, but only the sulfated one acted as nanoparticle drug stabilizers. Interestingly, the excitation and emission fluorescence spectrum of R595 (FIG. 2D) and of R555 (data not shown) showed small shoulders in 520 nm and 550 nm, a common pattern of cyanine dyes. Additionally, the emission curve of these dyes resembles cyanine dyes type of fluorescence around 620 nm and 650 nm in lower intensity.

We performed ¹H-NMR spectroscopy (FIG. 2E) and observed dramatic peak broadening which can indicate the formation of multiple products or a polymerization process. In addition, the aromatic region was shifted to lower ppm values (from ˜8.5 to ˜7.2 ppm). Protons close to the nitrogen of the indole at 4.5 ppm completely disappeared, indicating that they participate in the reaction with the bicarbonate buffer, likely in an oxidation step. In addition, alkyl hydrogens in the 1.2 ppm region increased dramatically and broadened, again implying multiple products. MALDI-TOF analysis confirmed our initial assumption and showed multiple peaks with repeating intervals increasing in size, supporting a polymerization reaction of the indoliums (data not shown).

We searched the literature for similar reactions and found strong resemblance with the polymerization process of dopamine into PDA (Liu et al., 2014b). In PDA as well, a colorless single monomer in a weak basic buffer yields a dark colored mixture of oligomers/polymers without initiators or catalysts. PDA is a useful biomaterial made from a mixture of oligomers with an undetermined structure which is used for coating almost any type of nanomaterial. Therefore, even though R595 is a mixture of oligomers, and the structure is not entirely clear, we decided to evaluate its properties and functions, as there are many advantages for PDA-like reactions. First, they are cheap, easily prepared and highly scalable; and second, they can be categorized as completely “green” as they are made of minimal components (single monomer in aqueous basic buffer) and do not include any hazardous solvents and auxiliaries.

As the automated discovery phase was performed in room temperature and in low volumes, the synthesis of R595 was further optimized and scaled up to larger volumes. We found that the optimal conditions for R595 synthesis was using 5 mg/ml of the indolium monomer, In820, in 0.1M of bicarbonate buffer, at 90° C. for 4 h under mixing. Similar results can be obtained in room temperature but with at least 48 h of incubation.

We scaled up the nanoprecipitation process of trametinib and nilotinib in R595 solution, characterized them with DLS and found nanoparticles with 50 nm and 70 nm diameter respectively (FIG. 2F). To improve nanoparticles preparation, we sought to automate the purification step as well. Previously, purification processes were mostly described with the use of centrifuges or dialysis, which are difficult to automate. We established a size exclusion chromatography method which is highly used for protein and DNA purifications, based on Sephadex G25 desalting columns (PD10). We show that the formed nanoparticles are smaller with a lower polydispersity index (PDI) compared to our centrifuge protocol nanoparticles, confirming our hypothesis that the PD10 is a compatible modification for the automated workflow (data not shown). Trametinib-R595 nanoparticles purified by PD10 were also imaged with SEM and found to have round shaped nanoparticles with diameter size of 50 nm (FIG. 2G). This was further confirmed by cryogenic TEM (Cryo-TEM) (data not shown).

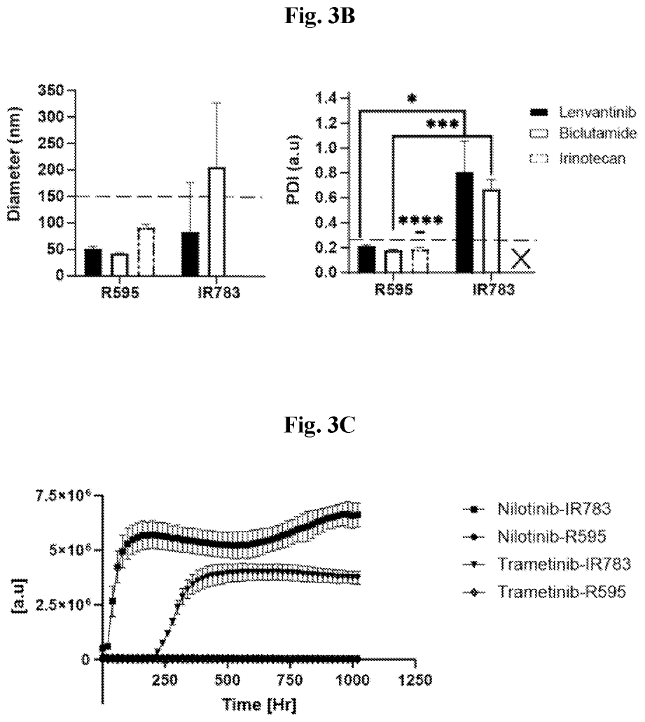

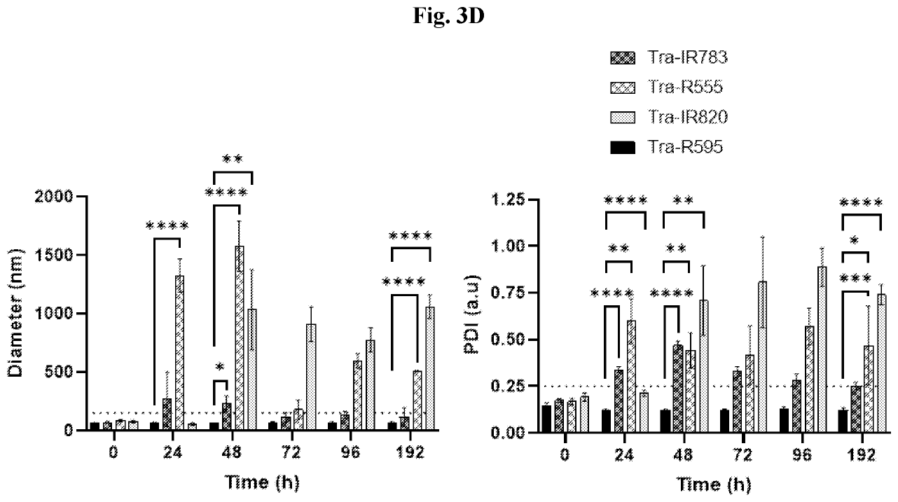

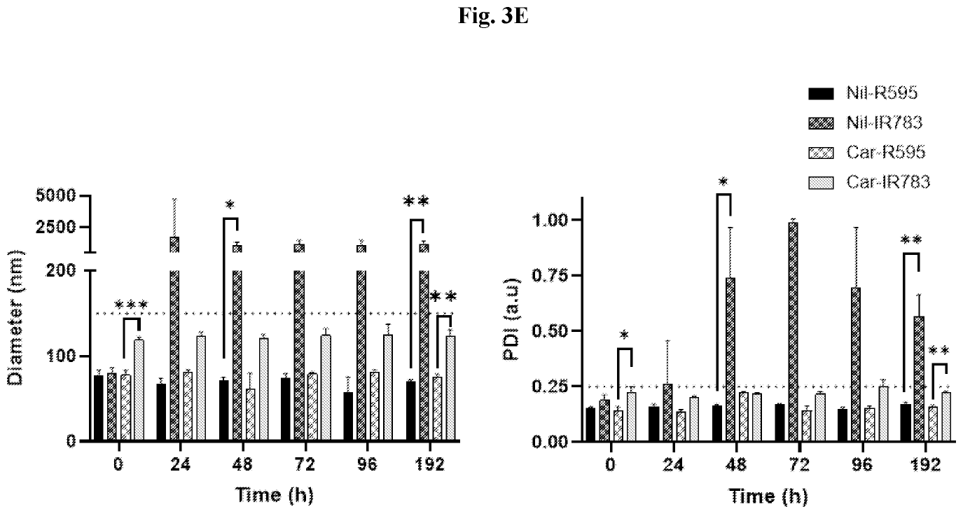

R595 is an ultra-stabilizer. We further tested R595 as a nanoparticle stabilizing agent and compared it to a panel of known nanoparticle forming stabilizers and solubilizers. In this experiment, we attempted to form a water dispersion of nilotinib by using a panel of detergents, polymers, and lipids that had previously been used to stabilize hydrophobic drug nanocrystals or dissolve them. We found R595 to be the only stable water dispersion agent with no sign of precipitation at equal weight ratios (FIG. 3A). We also compared the stabilization ability of R595 to IR783, previously reported as a superior nanoparticle stabilizer. We characterized suspension of lenvatinib, bicalutamide and irinotecan, which failed to form nanoparticles with IR783 but formed stable nanoparticles with R595 (FIG. 3B). Interestingly, irinotecan failed to co-precipitate with IR783 but did co-assemble with R595 into nanoparticles. All R595 nanoparticles had particle sizes lower than 150 nm, and low PDI below 0.25. We defined these values as thresholds parameters for optimal and stable suspension which also exploits the enhanced permeability and retention effect at the tumor site.

We then sought to characterize the kinetic stability of the nanoparticles using the AIE properties of the drugs. We selected AIEgenic drugs nilotinib and trametinib and evaluated their aggregation profile with fluorescent microscopy time lapse movies. We performed two image analysis measurements: 1) the number of aggregates per well; and 2) the total fluorescence signal in the region of interest (ROI). This enables accurate quantification of aggregates and changes in size which can inform on real time aggregation processes. We found that both drugs aggregated overnight with the IR783 dye but remained completely stable with R595 (FIG. 3C). Nilotinib-IR783 started aggregation within 40-60 min while trametinib began at 250 min. Also, nilotinib-IR783 had 4-fold fewer number of aggregates than trametinib, but they were 2-fold larger in size (sum area). Trametinib-IR783 aggregation process started with rapid formation of homogenous microparticles which grew slightly, whereas with nilotinib a small number of large aggregates was formed (FIG. 1D).

Next, we compared the long-term stability and shelf-life of R595 nanoparticles to other dye stabilizers. We found that within the first 24 hours, nanoparticles made with R595 as a stabilizer were superior to R555 and IR783, while IR820 was a better stabilizer than IR783 (FIG. 3D). When compared to all dyes at 192 h, R595 proved to be an ultra-stabilizer, with constant PDIs and size values. In fact, we did not observe a change in size or PDI over a period of 6 months. We also compared the unstable IR783 nanoparticles of nilotinib with carfilzomib and found a long-term stability of over a week with R595 compared to less than 24 h with IR783 (FIG. 3E). Even though carfilzomib-IR783 was within the desired range of nanoparticles size i.e., under 150 nm (118±2.65 nm), the nanoparticles had large PDI (0.22±0.020). R595 nanoparticles were 50% smaller (77±4.63 nm) and mono-dispersed (0.14±0.016), supporting the superiority of R595 as an ultra-stabilizer.

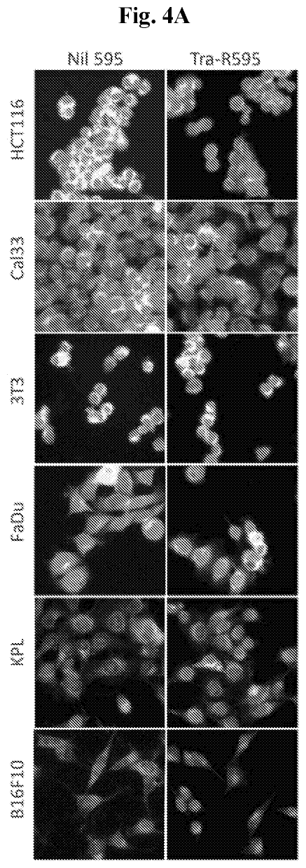

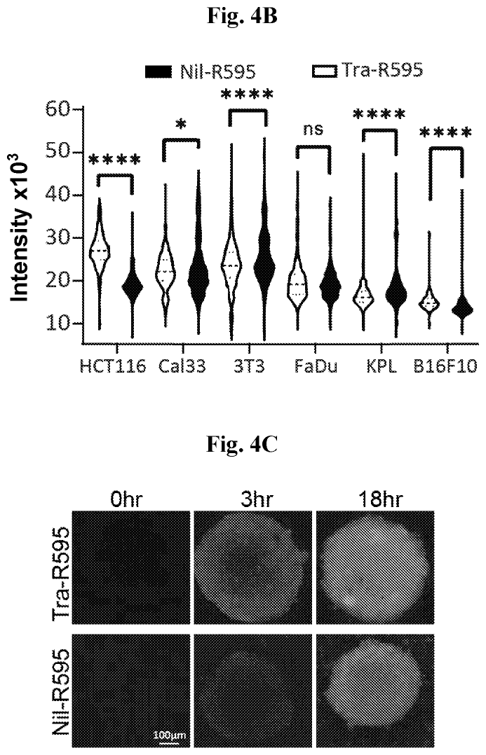

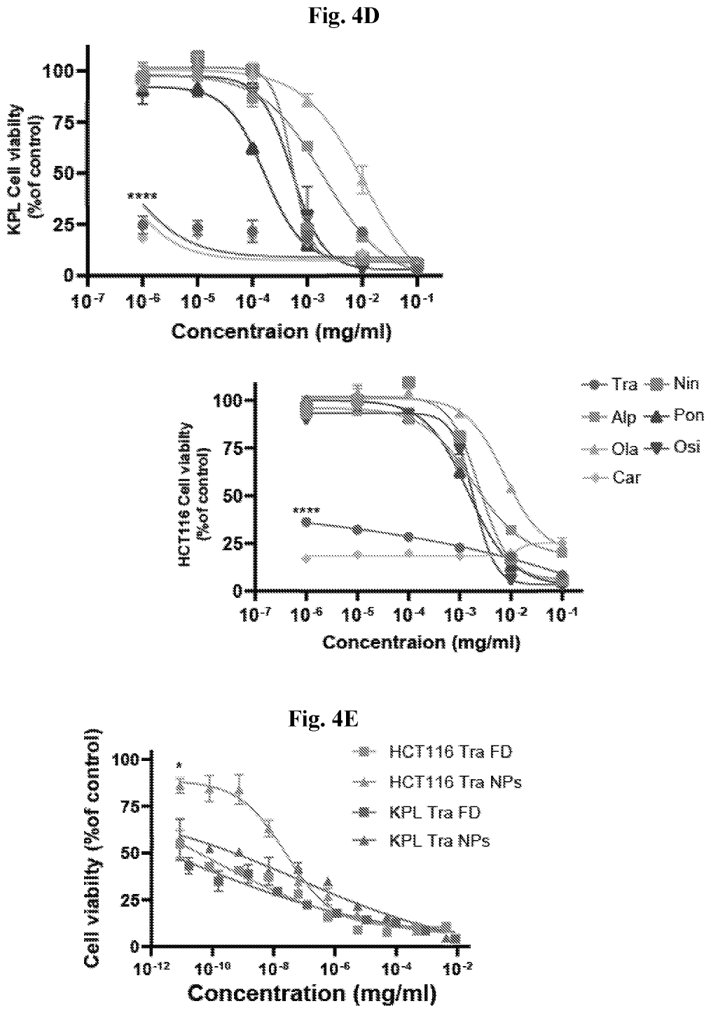

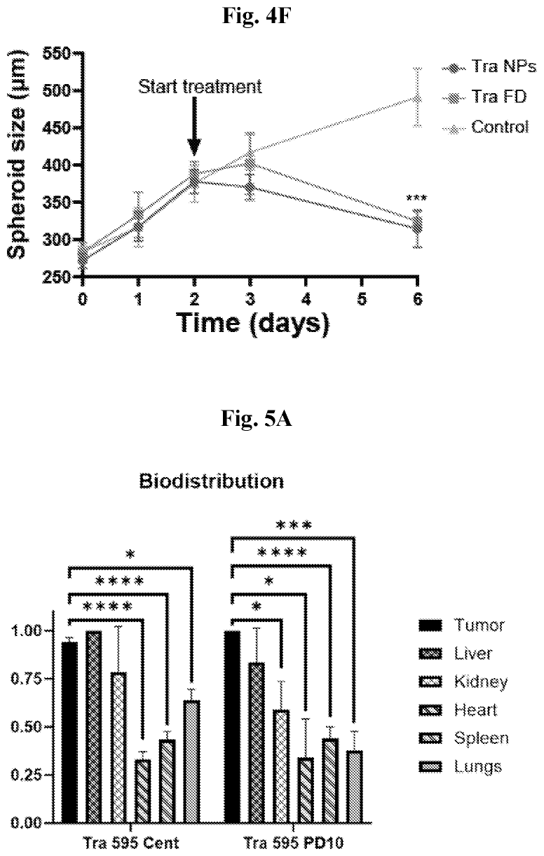

In vitro uptake and efficacy. Previously, IR783 based nanoparticles were reported to exhibit differential cellular uptake hypothesized to be mediated by CAV1 (Shamay et al., 2018). To evaluate the differential uptake of our newly developed nanoparticles, we incubated R595 nanoparticles of trametinib or nilotinib with six different cell lines and visualized uptake with automated fluorescence microscopy. Indeed, we observed differential uptake of the different nanoparticles (FIG. 4A). A common phenotype of grainy intracellular fluorescence was observed within vesicles resembling endosomes. Quantification of the differential uptake clearly showed that 3T3 and HCT116 have the highest uptake, followed by Cal33, FaDu, KPL and B16F10 cells with the lowest amount (FIG. 4B). Interestingly, nilotinib nanoparticles had significantly more uptake to HCT116 cells than trametinib but the opposite was seen in KPL. We also observed efficient uptake of sorafenib-R595 nanoparticles compared to trametinib-R595 in both endothelial and KPL cells (data not shown). We next evaluated the penetration of nanoparticles into 3D tumor spheroids. We hypothesized that AIEgenic drugs will be fluorescent when they are cell bound (aggregated either in lysosomes or in the endoplasmic reticulum) as opposed to drugs or nanoparticles in solution, in the same manner that nuclear staining with Hoechst is effective without washing out the unbound dye probe. We incubated KPL spheroids with R595 nanoparticles of nilotinib and trametinib and evaluated the penetration of the nanoparticles via live imaging fluorescent microscopy (FIG. 4C). Within 3 hours it was already clear that trametinib-R595 nanoparticles penetrated faster to the spheroids, which was further quantified by confluence and intensity measurements of the accumulated drugs. High concentration trametinib reached maximum confluence value within 7 hours while nilotinib nanoparticles of equal concentration—only after 12 hr, demonstrating the differential nature of nanoparticle uptake. We conducted a similar experiment on HCT116 spheroids and after 15 hours of treatment we observed complete penetration throughout the spheroids of both R595 and the AIEgenic drugs (data not shown). This result was encouraging as some nanoparticles were previously reported to have poor penetration in tumor spheroids. We then performed a small anti-cancer drug-screen for HCT116 and KPL with 7 drugs and observed the highest potency for trametinib and carfilzomib (FIG. 4D). As these cells are KRAS driven it is not surprising that the MEK1/2 inhibitor trametinib will show strong activity. Carfilzomib is a proteasome inhibitor used mainly in multiple myeloma and some types of lymphomas. We then compared the activity of the trametinib nanoparticles to the free drugs in 2D and 3D cell cultures. The activity of the nanoparticles was comparable to the free drugs in all concentrations in the KPL 3D model but had lower activity in low concentrations on HCT116 spheroids (FIG. 4E). This might imply that the HCT116 spheroids have a more compact arrangement than the KPL ones rendering it difficult for the nanoparticles to penetrate the structure. Even though the cell viability assay shows decreased activity, a similar efficacy profile was shown when the spheroid size was the primary readout. A representative analysis is shown in FIG. 4F.

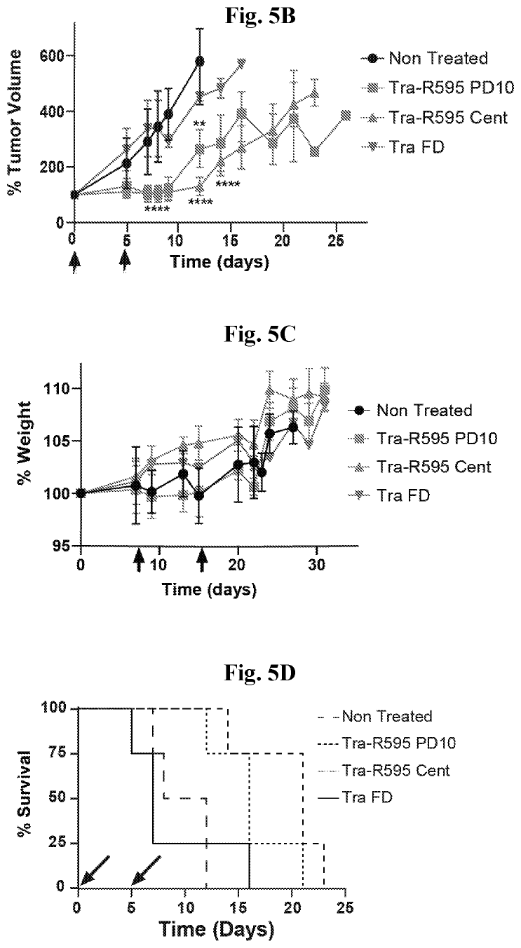

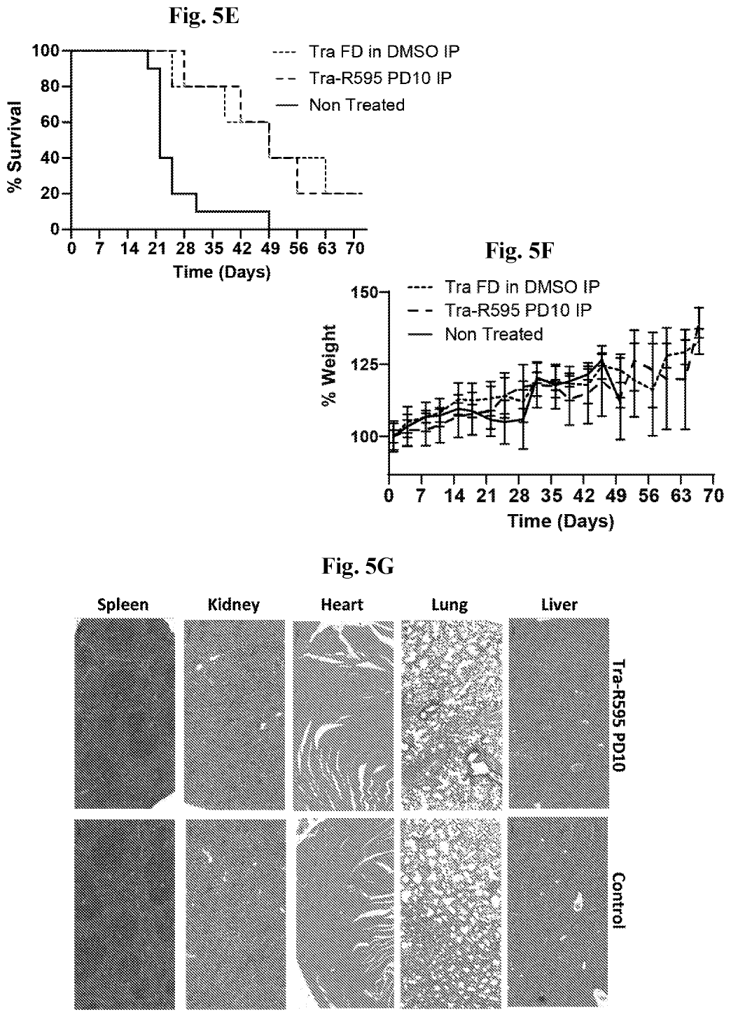

In vivo safety and efficacy of R595 nanoparticles. Next, we evaluated the biodistribution, safety and efficacy of R595 nanoparticles in vivo. For the biodistribution studies we chose HCT116 subcutaneous xenografts model of colon cancer which in vitro showed substantial uptake and efficacy. When tumors reached an average sum volume of ˜600 mm³ mice were IP injected with trametinib-R595 nanoparticles purified via centrifuge or PD10. Fluorescence imaging of harvested organs 24 h after injection revealed localization specifically within tumor tissue and the liver as the second organ of distribution with some advantage to the PD10 purification method (FIG. 5A). Interestingly, there was no significant accumulation in the lungs as described for IR783 (Shamay et al., 2018). We then tested the anti-cancer efficacy compared with FD given PO and the two different purification methods of trametinib-R595 nanoparticles. As it is already established that a daily administration of trametinib is highly effective in the FD, we chose only two single administrations on days 0 and 5 to demonstrate the benefit in accumulation and sustained release of our nanoparticles. As shown in FIG. 5B, the nanoparticles of both purification methods were significantly potent as compared to the FD (Day 12, P=0.0042). By comparing the mice's weights during the experiment, we did not detect any statistically significant differences, suggesting that trametinib-R595 is as safe as the free drug, yet much more effective (FIG. 5C). We analyzed survival using Mantel-Cox test on Kaplan-Meier curves (FIG. 5D) and found significant benefit for the treatment groups (P=0.0127). There was no statistical difference between PD10 and centrifuge. Our second experiment tested the safety and efficacy of the trametinib-R595 using a more aggressive KRAS driven model of lung metastases, in which KPL cells were injected IV to develop multiple tumors in the lungs. Mice were treated with either a nanoparticles formulation or free drug (in DMSO) via IP injections. As found, both trametinib-R595 nanoparticles and FD had a significant and comparable anti-cancer activity (FIG. 5E), while both had no dose limiting toxicities as observed with weight changes (FIG. 5F). In both treatments the median survival was extended up to 49 days, compared with median survival rate of 22 days in the non-treated mice (P=0.0042). As opposed to the marked difference between nanoparticles and free drug in the HCT116 model, the lack of difference between these groups in the KPL model might have two possible explanations. One such explanation is the rise of acquired resistance to the MAP kinase pathway which is known to occur in this cancer cell type and is unrelated to targeted delivery process; and the other explanation might be the lack of nanoparticle uptake which was significantly lower in this model in vitro as opposed to the HCT116 model (FIG. 4B). For long term toxicity and safety experiments, we administered mice with trametinib-R595 nanoparticles purified with PD10, twice a week for two months, and followed their weight, vital signs and activity. We then harvested histology samples of liver, lung, heart, kidney, spleen, and skin tissues from treated mice, and compared them with non-treated mice (FIG. 5G). We could not detect any meaningful changes in histology between the groups, or any statistically significant weight loss throughout the course of this long-term treatment (data not shown).

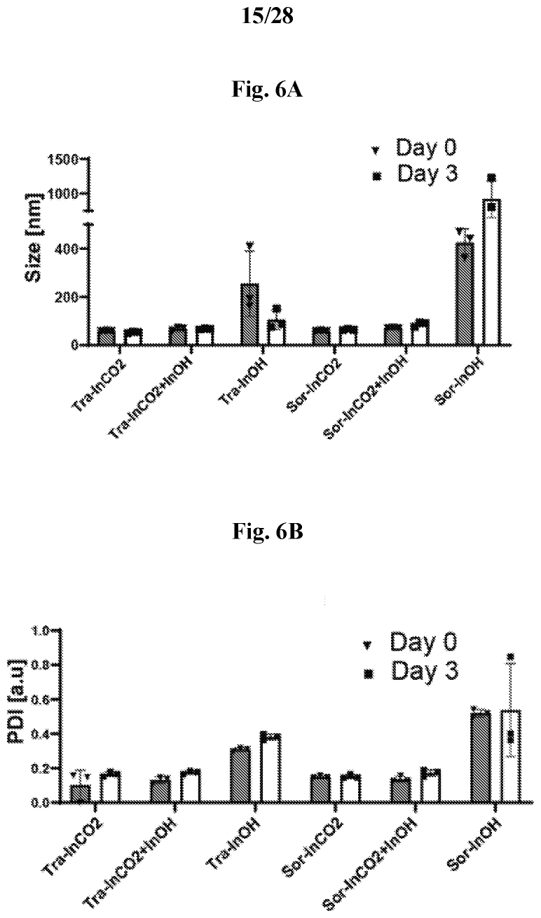

Additional indolium based monomers. Investigation of additional indolium-based monomers for hydrophobic drug stabilization: This study also assessed the efficacy of two indolium-based monomers that lack a sulfuric acid group for stabilizing hydrophobic drugs. The monomers are identified as 1-(2-hydroxyethyl)-2,3,3-trimethyl-3H-indolium iodide, referred to hereafter as In—OH, and (5-carboxypentyl)-1,1,2-trimethyl-1Hbenz[e]indolium bromide, henceforth abbreviated as In—CO2. These indolium-based monomers were subjected to a 24-hour incubation in bicarbonate buffer at a temperature of 90° C., using a methodology analogous to the synthesis of R595. The monomers were tested both individually and combined in a 1:1 ratio (comprising 50% In—CO2 and 50% In—OH) to assess their efficacy in stabilizing trametinib (Tra) and sorafenib (Sor). Our results, substantiated by DLS size and PDI measurements (FIGS. 6A-6B), revealed that the In-CO2-based polymer was effective in stabilizing both trametinib and sorafenib; the In—OH-based polymer was effective only for trametinib; and the 1:1 mixture of In—CO2 and In—OH was effective as In—CO2 in stabilizing both drugs as well.

### Study 2. Polydopamine Copolymers for Stable Drug Nanoprecipitation

### Materials and Methods

Materials and reagents. All non-drug chemicals were purchased from Sigma Aldrich (St. Louis, MO). DMSO was purchased from Carlo Erba (Emmendingen, Germany). All drugs were purchased from LC-Laboratories (Woburn, MA) and MedChemExpress.

Polymerization process. Conditions matrix: dopamine (Sigma Aldrich), L-dopa (Sigma Aldrich), norepinephrine (Sigma Aldrich), serotonin (Holland Moran), tyramine (Sigma-Aldrich), and tryptamine (Holland Moran) were dissolved in DDW to a concentration of 4 mg/ml. The polymerization solutions, except for NaOH, were prepared in advance and diluted by half when mixed with the water dissolved monomers. NaOH (Biolab, 10⁻³M,) was diluted to 10⁻⁵M with the monomers. Initial concentrations: TRIS buffer (Sigma-Aldrich)—0.01M, sodium bicarbonate buffer (Biolab)—0.2M, hydrogen peroxide (Merck)—30% in DDW.

Co-monomers matrix. The reactions were carried out in sodium bicarbonate buffer 0.1M. Polyethylene glycol-SH (PEG-thiol, Mn=1 kDa, Creative PEGWorks), 1,1,2-trimethyl-3-(4-sulfobutyl)benz[e]indolium, inner salt (In820, Sigma-Aldrich), 2,3,3-trimethyl-1-(4-sulfobutyl) indolium (In783, Sigma-Aldrich). 1,2,3,3-tetramethyl-3H-indol-1-ium iodide (In783-nosulfate, Holland Moran), N-(2-hydroxypropyl) methacrylamide (HPMA, Sigma-Aldrich), and sodium lauryl sulfate (SDS, Spectrum) were dissolved to a concentration of 2 mg/ml, and polyethylene glycol methyl ether methacrylate (PEG-MA, Mn=2 kDa, Sigma-Aldrich) in a final concentration of 7.5 mg/ml. 200 μl of the monomer-co-monomer (50:50% v) solution were added to wells in a 96-well plate and incubated covered at room temperature for 4 hours.

Polymerization of copolymers: for the dual copolymers, 4 mg of each monomer were dissolved in 1 ml of sodium bicarbonate buffer (0.1M) and mixed, final concentration of each monomer—2 mg/ml. For the triple copolymers, 6 mg of each monomer were dissolved in 1 ml of sodium bicarbonate buffer (0.1M) and mixed, final concentration of each monomer—2 mg/ml. The copolymers were shaken at 800 rpm for 4 hours at room temperature, stored in a dark place, and aged for 7 days before used.

Absorbance measurements. Absorbance and emission spectra were evaluated with Synergy H1 (BioTek®) plate reader. Absorbance was measured every 30 min and emission spectra were measured 4.5 hr from the beginning of the polymerization process, each polymer was excited according to its absorbance spectra.

ATR-FTIR. Samples were lyophilized by Freeze zone 2.5 (Labconco, USA) at −54° C., 0.01 mbar for 48 hours. Transmittance was measured in Bruker's Fourier transform infrared (FT-IR) Alpha at 400-4000 cm⁻¹ to identify the chemical signature of the polymers. The measurements were done at the Chemical and Surface Analysis Lab, at The Schulich Faculty of Chemistry, the Technion.

Imaging fluorescent drug aggregates with automated microscopy. To image fluorescent drug aggregates, 10 μl of nilotinib were added (1 mg/ml) to 60 μl bicarbonate buffer (0.1M) and 20 μl of the copolymers to wells in 96-well plate. The wells were imaged in the DAPI channel Lionheart (BioTek®) with a 10 ms exposure 10% digital gain, 100% LED intensity and image based autofocus. AIE effect quantification was done by calculating the relative confluence of blue emission in each well compared to nilotinib aggregates in water.

Preparation of nanoparticles. Drugs dissolved in DMSO (Carlo Erba, 10 mg/ml) were added under slight vortex to aqueous dye solution, buffered with 0.1M sodium bicarbonate. IR783 (Sigma-Aldrich) and the polymers' concentrations were 2 mg/ml. The solution was centrifuged (30,000 g, 15 min, room temperature) and the pellet was resuspended in 1 ml of DDW or purified using a PD-10 desalting column (GE Healthcare, Chicago, IL), and 1.7 ml of eluent was collected. If the particles were cleaned in centrifuge, the solution was sonicated using Sonics' Vibra-cell ultrasonic processor (20% amplitude, 3 seconds pulses) until homogenous.

Preparation of nanoparticles with drug pairs. Drugs dissolved in DMSO (Rap [rapamycin], Pon [ponatinib] and Tra [trametinib]10 mg/ml) were added at a 1:1 mass ratio under slight vortex to aqueous copolymer or R595 mixture solution, buffered with 0.1M sodium bicarbonate. IR783 (Sigma-Aldrich) and the polymers' concentrations were 2 mg/ml. The solution was centrifuged (30,000 g, 15 min, room temperature) and the pellet was resuspended in 1 ml of DDW or purified using a PD-10 desalting column (GE Healthcare, Chicago, IL), and 1.7 ml of eluent was collected. If the particles were cleaned in centrifuge, the solution was sonicated using Sonics' Vibra-cell ultrasonic processor (20% amplitude, 3 seconds pulses) until homogenous.

Characterization of nanoparticles. DLS measurements were conducted using a Zetasizer Nano ZS (Malvern Panalytical, Malvern, UK).

Drug loading. nanoparticles diluted 1:10 in acetonitrile:ethanol (50:50) solution were measured with ultra-performance liquid chromatography (Acquity arc UHPLC, Waters Corp, MA) using a CORTECS C18 (4.6×50 mm 2.7 μm) column.

HR-SEM. 2 μL from the nanoparticles solution were applied to a silicon wafer and placed in a desiccator under vacuum for 72 h for dehydration. High resolution scanning electron microscopy (HR-SEM) imaging was performed by the Technion EMC—The Electron Microscopy Center on a Zeiss Ultra Plus high-resolution SEM, equipped with a Schottky field-emission gun. Specimens were imaged at acceleration voltages of 1.3 kV and a working distance of 2.4 mm. The Everhart Thornley (“SE2”) secondary electron imaging detector was used.

Cell cultures. 3T3 cells were provided by the Heller lab at MSKCC, and HCT 116 cells were a kind gift provided by the lab of Moshe Elkabets, Ben-Gurion University, Israel. The cells were cultured in DMEM supplemented with 10% FBS, 2 mM L-glutamine, penicillin G Sodium Salt: 100 units/mL and streptomycin sulfate: 0.1 mg/mL (pen-strep). All cells were incubated at 37° C. with 5% CO₂ and 65% humidity.

Polymers toxicity assay in 2D. 3T3 Cells were seeded in 96 well plates at a 30% confluency (6000 cells per well) and allowed to adhere for 24 h. Polymers were purified with Sephadex G25 columns, nanoparticles were suspended in DDW and added to the wells. Untreated cells (control) were used to establish 100% viability. The effect on cell viability was measured with MTT (5 mg/ml, Glentham Life Sciences, UK).

Cell viability assay in 2D. HCT 116 cells were seeded in 96 well plates at a 30% confluency (5000 cells per well) and allowed to adhere for 48 h. All drugs were dissolved in DMSO, nanoparticles were suspended in DDW and added to the wells. Untreated cells (control) were used to establish 100% viability. The effect on cell viability was measured with Promega® CellTiter-Glo® (CTG) 2D cell viability assay.

Cell viability assay in 3D. HCT 116 cells were seeded in 96 well round bottom ultra-low attachment plates (1000 cells per well) and allowed to form spheroids for 72 h. All drugs were dissolved in DMSO, nanoparticles were suspended in DDW and added to the wells. Untreated cells (control) were used to establish 100% viability. The effect on cell viability was measured with Promega® CellTiter-Glo® (CTG) 3D cell viability assay.

### Results and Discussion

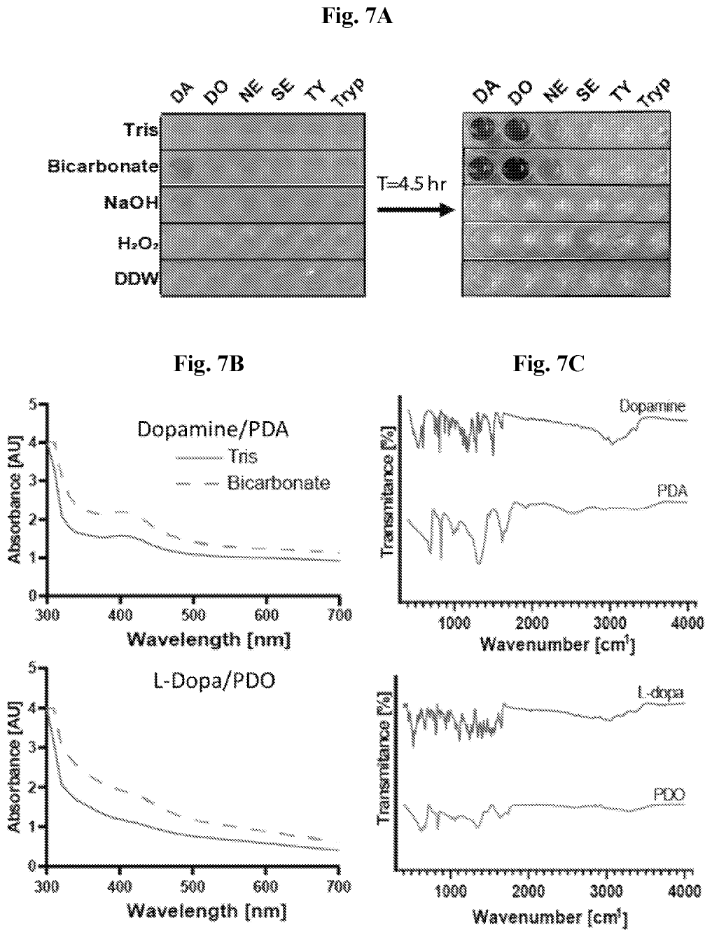

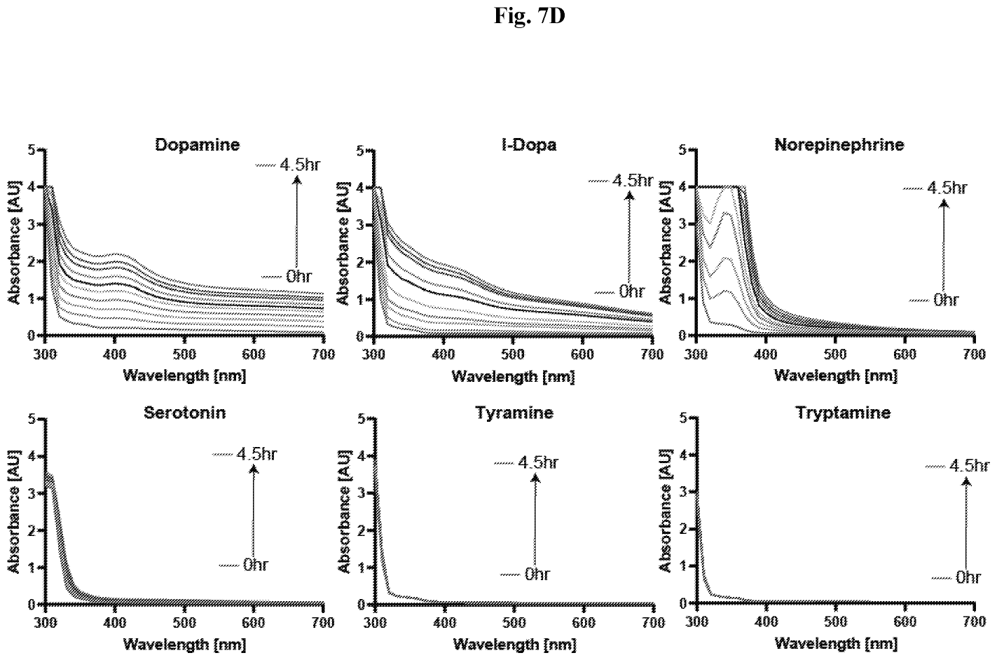

Finding the optimal conditions for polymerization. Six catecholamines and related aromatic amine monomers were tested in five different conditions and DDW as control (FIG. 7A). Besides dopamine, we tested L-dopa, norepinephrine, serotonin, tyramine and tryptamine. All monomers were at a final concentration of 2 mg/ml as previously described (Wei et al., 2010). Tris and sodium bicarbonate buffers (pH 9) are well-known alkaline buffers for polymerization of catecholamines. They were compared to a NaOH solution at the same pH level and a hydrogen peroxide solution, to test the effect of oxidation. In order to evaluate the progression of the reactions, the absorbance of the solutions was measured over 4.5 hours. As expected, the most dominant change was in Tris and bicarbonate buffers (FIG. 7B). The process, observed by the change in color, was significantly faster in the sodium bicarbonate buffer than in tris buffer. Therefore, we chose this buffer for the copolymerization process as well. We measured the emission spectra of dopamine (ex. at 400 nm) and L-dopa (ex. at 420 nm), they match the expected peaks of catecholamines after polymerization. ATR analysis of poly(dopamine) and poly(L-dopa) (PDO) in comparison to the monomers (FIG. 7C) revealed peaks at 2936 cm⁻¹ and 3335 cm⁻¹, which correlate to the primary amine in dopamine. The decrease in the transmittance after the polymerization indicates the expected ring closing. The change in the transmittance at 2400-3500 cm⁻¹ hints the disappearance of the carboxylic acid in L-dopa. It was suggested that part of its oxidation process involves losing the carboxylic acid (Yang et al., 2018).

The change over time in the absorbance spectra of the different monomers in sodium bicarbonate buffer is shown in FIG. 7D. Dopamine and L-dopa polymerization process and change of color are faster and more apparent than norepinephrine and serotonin, while tyramine and tryptamine did not change color at all. The absorbance spectra of PDA and PDO correlate to the expected spectra for poly(catecholamine)s. We hypothesized that the ability to polymerize under these conditions relates to the monomers' structure. Dopamine, L-dopa and norepinephrine are all catecholamines and have two hydroxyl groups on the benzene ring, while serotonin and tyramine have only one hydroxyl group, and tryptamine has no OH groups. The polymerization process is not fully understood but known to involve the oxidation of the monomers and formation of a quinone which can only happen with two hydroxyls.

Optimal copolymer for hydrophobic drugs stabilization. Poly(catcholamine)s are hydrophobic polymers and will not be able to stabilize hydrophobic drugs because they precipitate on their own in water. Therefore, we sought a co-monomer that prevents the precipitation of the copolymer, as well as stabilizes nanoparticles of hydrophobic drugs prepared by nanoprecipitation. To find the optimal monomer combination, suitable for drug stabilization in the nanoprecipitation method, a panel of co-monomers was tested. The panel included known PDA co-monomers and modifiers such as PEG-SH (Zhang et al., 2020), PEG-methacrylate (Zhang et al., 2017) and HPMA (Prostota and Coelho, 2013). In addition, we tested sulfated indolium molecules which are known to react with aldehydes in fluorescent dye synthesis (Wang et al., 2021). We hypothesized that if PDA can react with thiols, amines and methacrylates, it might be able to react with indoliums.

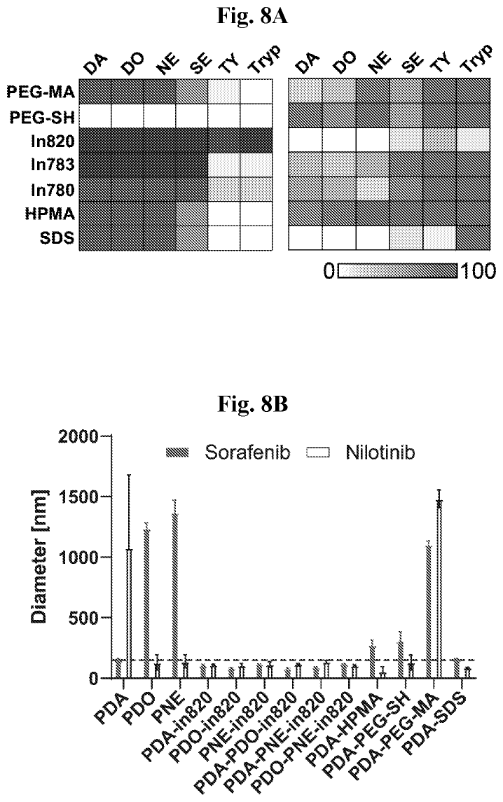

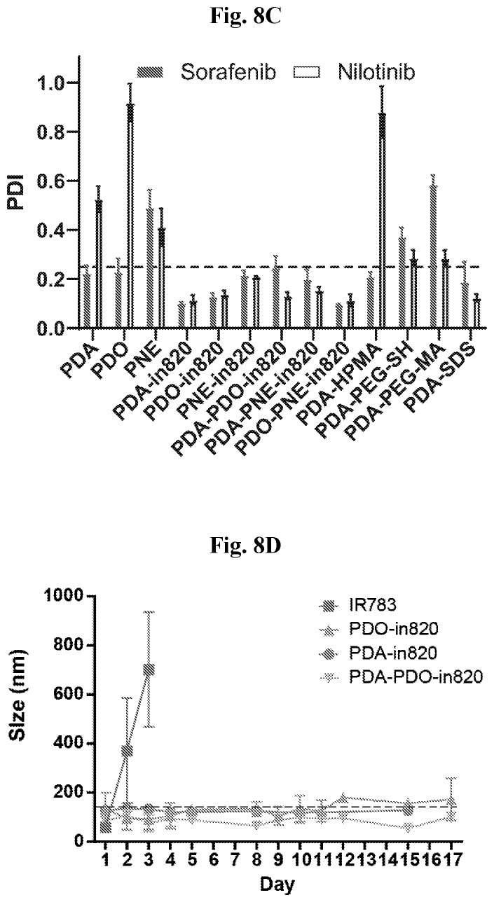

We first tested the copolymer's ability to stabilize nilotinib, a hydrophobic kinase inhibitor, by utilizing its AIE properties. AIE refers to increased fluorescence emission in the aggregated state over the soluble state (Würthner, 2020; Wu et al., 2019; Shamay et al., 2018). Based on Study 1, we precipitated nilotinib with bicarbonate buffer and the different copolymers, in a 96-well plate and measured the relative surface area (confluence) that is blue under DAPI filter, where higher confluence correlates with higher particle size. An illustration of the colors formed after 4 hours polymerization is shown in FIG. 8A (left panel). Some of the wells turned brown due to the catecholamines polymerization reaction, while wells containing In820 and In783 turned purple and red, respectively, since they are precursors for dyes. In780 is also a dye precursor, and its reaction in the bicarbonate buffer with and without the catecholamines formed different colors. The confluence was normalized to nilotinib aggregates in sodium bicarbonate buffer (0.1M) and is shown in FIG. 8A (right panel). The lowest intensity measured was in copolymers containing In820 and SDS, indicating their superiority over PEG and HPMA. SDS containing copolymers resulted in aggregation after nanoparticle purification process, thus we decided to further study In820 copolymers with different catecholamines and not SDS.

We further validated these results and measured the ability of different PDA copolymers to stabilize sorafenib and nilotinib with DLS (FIGS. 8B-8C). Like nilotinib, sorafenib is a hydrophobic multi-kinase inhibitor used extensively in nanoparticles made via nanoprecipitation; however, it is more likely to form stable nanoparticles than nilotinib. The homopolymers of dopamine, L-dopa and norepinephrine were not able to form stable nanoparticles with sorafenib and nilotinib, and precipitated on their own. Surprisingly, PEGylation of the polymers failed in stabilizing sorafenib but did stabilize nilotinib. The only copolymers which stabilized both drugs in nanoparticles smaller than 150 nm and with PDI around 0.25 were those containing In820 (FIGS. 8B-8C), which were also the only copolymers completely soluble in water. We focused on combinations without norepinephrine due to the slow polymerization kinetics. To further evaluate the co-monomers, we measured the stability of nilotinib nanoparticles stabilized by either IR783, PDO-In820, PDA-In820, or PDA-PDO-In820, over long time periods (FIG. 8D), and as found, while nanoparticles stabilized with IR783 aggregated within the first 24 h, nanoparticles stabilized by the In820 containing copolymers remained stable for more than two weeks. The optimal formulation with the smallest size and highest encapsulation ratio was PDA-PDO-In820. The encapsulation ratios with PDA-In820 and PDO-In820 were fairly low—only 45% and 12%, respectively, compared to 89% for the PDA-PDO-In820 nanoparticles, and their size remained lower than 150 nm after 17 days.

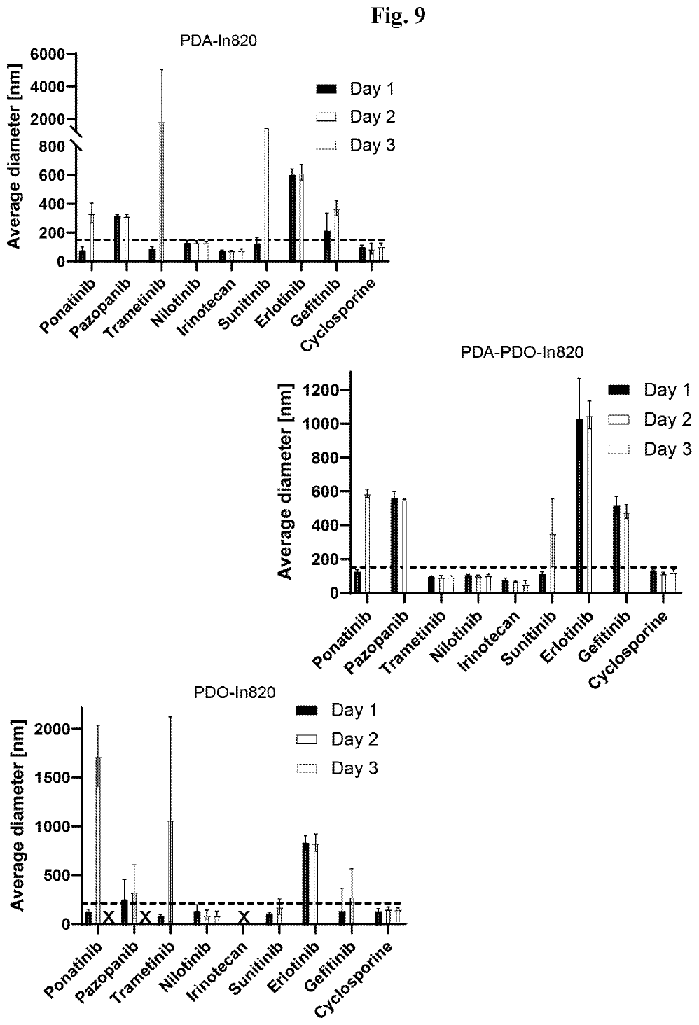

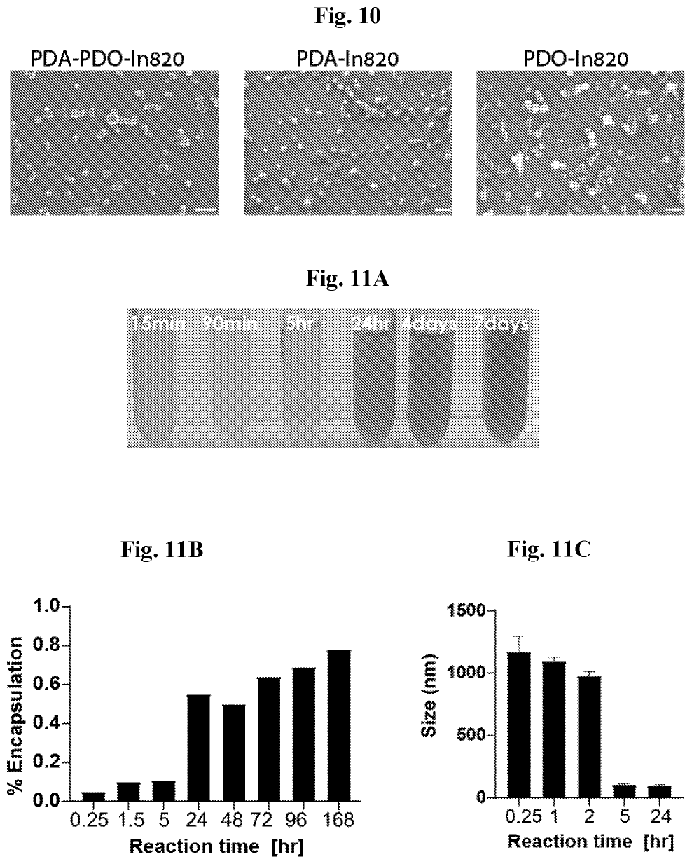

Then, we compared the three leading copolymers with 9 different hydrophobic drugs. We tested a panel of drug formulations with PDA-PDO-In820, PDA-In820 or PDO-In820 and monitored their stability over three days (FIG. 9). Drugs that were stable for two days were measured on the third day as well. Nilotinib and cyclosporine are the only drugs that were successfully stabilized by all three polymers for three days. PDO-In820 was the worst among the three copolymers. It was only able to stabilize nilotinib and cyclosporine for three days, while irinotecan did not form nanoparticles at all. The difference in the stabilization ability between PDA-In820 and PDA-PDO-In820 was seen in trametinib. The nanoparticles aggregated after two days when stabilized with PDA-In820 but remained stable for 3 days when stabilized by PDA-PDO-In820.

The final comparison was based on the morphology of the nanoparticles as captured by SEM imaging (FIG. 10). For this, we chose sorafenib as the model drug. For all three stabilizers the particles were spherical. Some aggregates were seen, probably due to the sample preparation and drying processes. The sizes measured in the SEM images were close to the expected sizes. The differences between the DLS and SEM measurements were less than 25%. Based on these results, we concluded that the morphology and size of the three formulations are the same.

Optimization of PDA-PDO-In820 synthesis. After we concluded that PDA-PDO-In820 can stabilize the largest variety of drugs, forms the most stable nanoparticles and has the highest encapsulation efficiency, we sought to further characterize it and optimize its synthesis. Since the structures of PDA and poly(L-dopa) are unknown, we did not aspire to identify the exact structure of the copolymer.

Different ratios of the three monomers were tested and showed similar stabilizing abilities. Therefor the simple 1:1:1 ratio was chosen. We tested both the size and relative drug encapsulation in the nanoparticles over time from the beginning of the polymerization process. We noticed the polymer requires an aging period of a week before it can form stable nanoparticles with high drug loading. The drug loading calculation was done for nilotinib nanoparticles (FIGS. 11A-11B). In the first two hours of the polymerization process the precipitation process is not efficient. Even though the polymer had dark color after the first 15 minutes, the solution was pale with total drug loading of ˜10%. After 24 hours, the nanoparticle suspension had a pale brown color with drug loading of 55%. Only after 7 days, we saw a significant increase in both the drug loading (78%) and the color of the suspension.

We also measured the size and stability of ponatinib nanoparticles with different reaction times (FIG. 11C). When formulating nanoparticles of ponatinib with freshly made copolymer (up to 5 hours from polymerization initiation), microparticles are formed instead of nanoparticles. Particles stabilized with PDA-PDO-In820 5 hours and 24 hours from the polymerization initiation were in the desired size range of below 150 nm.

During this process we also learned that different drugs form better particles by different purification methods. Nilotinib nanoparticles purified in centrifugation were not stable for more than a day, while their stability was maintained for 2 weeks when purified in a Sephadegx G25 PD10 desalting column. On the other hand, ponatinib only forms nanoparticles when purified in centrifugation.

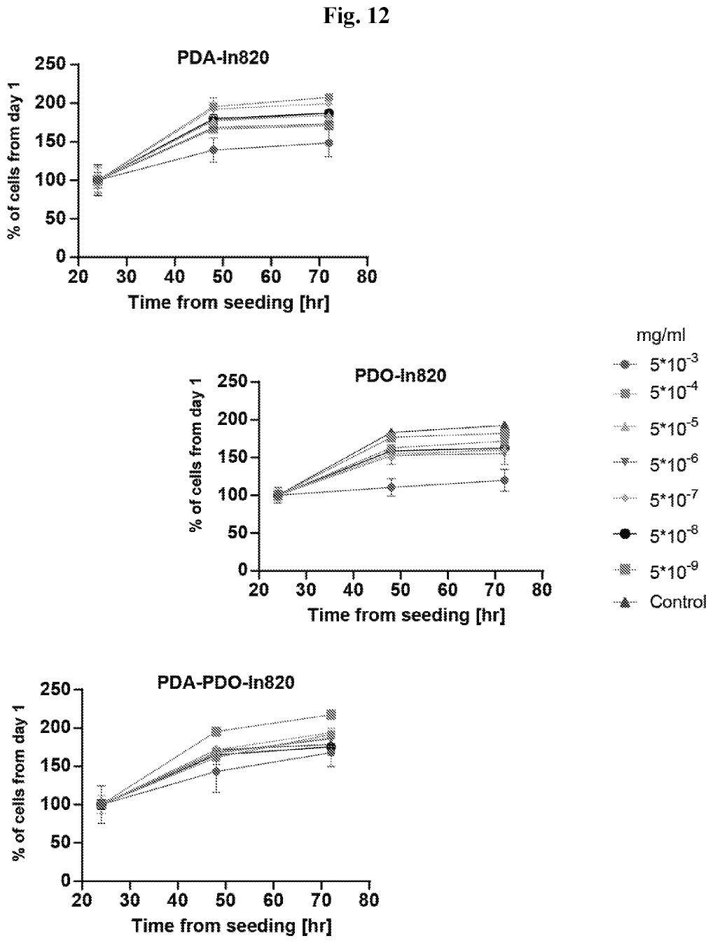

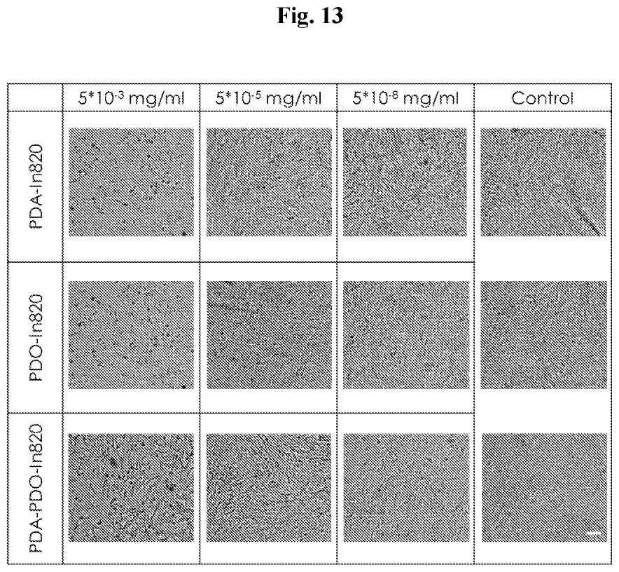

In-vitro toxicity and anti-tumor efficacy experiments. First, we tested the three leading copolymers for in-vitro cytotoxicity, using mouse embryonic fibroblast cells (3T3 cells) as a non-cancerous model (FIG. 12). The copolymers were purified with Sephadex G25 columns and were serially diluted in a 96-well plate seeded with the cells. We used two separate viability assays: MTT and an image-based cell counting method using automated microscopy. In the MTT assay we found no reduction in cell viability over control, even in the highest concentration of polymers (FIG. 12, upper and middle panels). As the image-based cell counting is non-invasive, we followed cell proliferation over time and found that in agreement with the MTT assay, the copolymers are not only non-toxic, but they are also non cyto-static and allow cell proliferation, even in the highest concentration (FIG. 12, lower panel). The cells morphology was not changed following incubation with the polymers, though at higher concentrations intracellular dark vesicles could be observed, indicating cellular uptake into the endo-lysosomal pathway (FIG. 13).

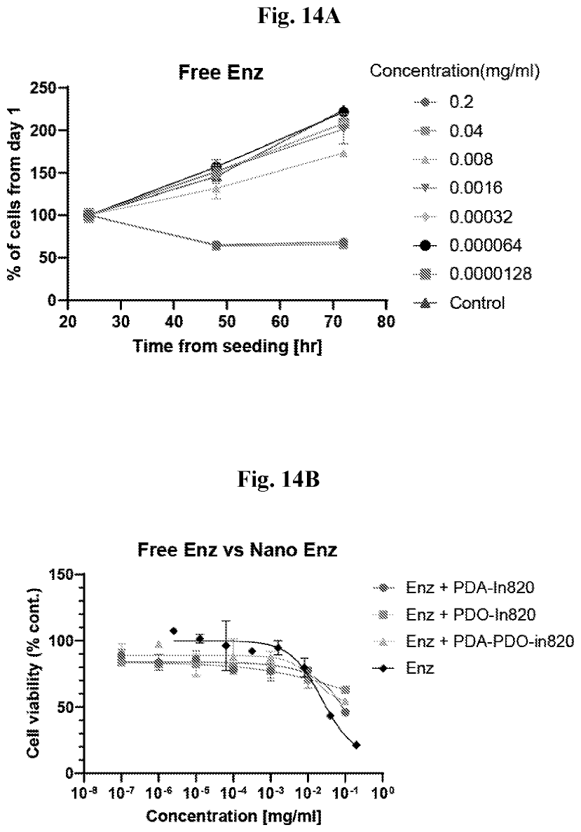

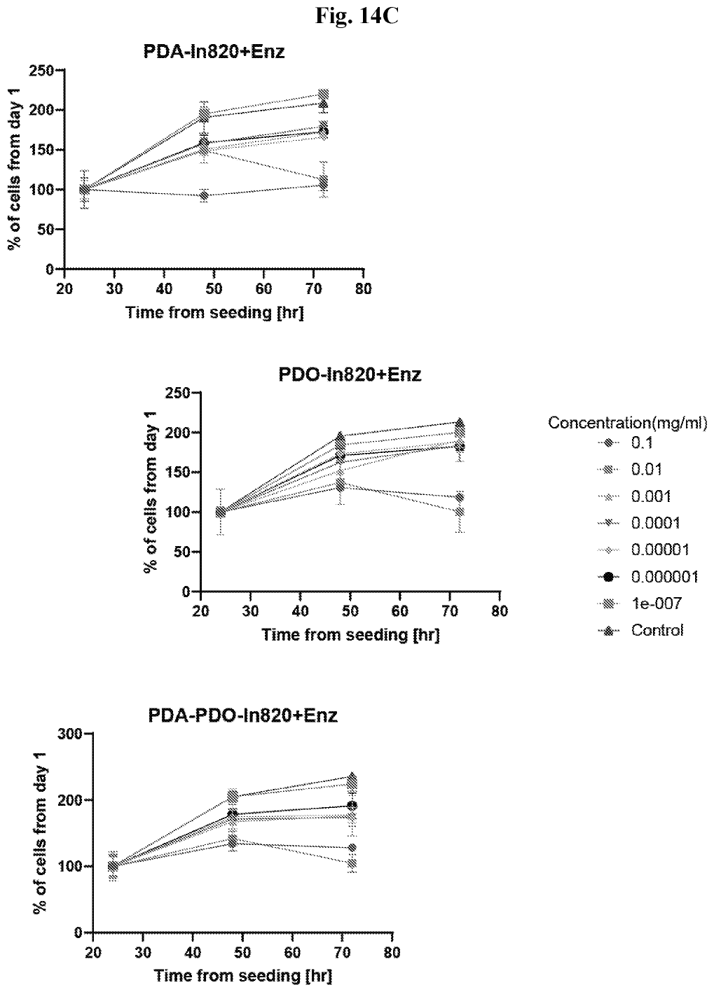

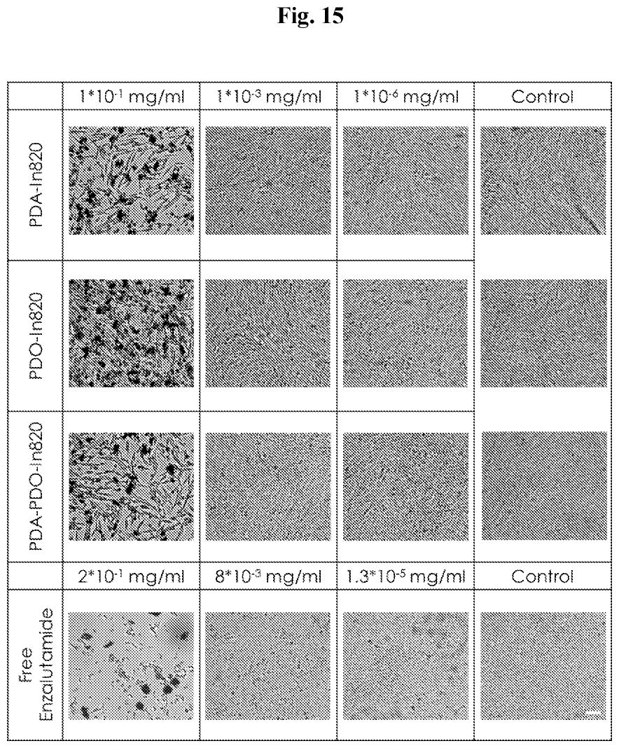

Enzalutamide nanoparticles were formulated and tested as non-toxic particles. It is a selective competitive androgen receptor inhibitor used for prostate cancer and is not expected to have toxicity in fibroblast cells which are not dependent on this receptor. Free enzalutamide had slight toxicity in high concentrations, that was attributed to the >5% DMSO in the medium. In high concentrations (0.1 and 0.01 mg/ml) of the nanoparticles, growth inhibition and cell death were observed (FIG. 14). Similar to the images in FIG. 13, intracellular dark vesicles were seen in the highest concentration and might have cause the observed cell death (FIG. 15).

Antitumor efficacy experiments were done on HCT116 cell line. Since the main driving mutation in this cell line is in the Ras proto-oncogene, it is known to be sensitive to trametinib. Sorafenib, a multi-kinase inhibitor, was also tested as a less efficacious drug. Both drugs were tested as free drugs and as nanoparticles stabilized by PDA-PDO-In820 on 2D and 3D in-vitro models.

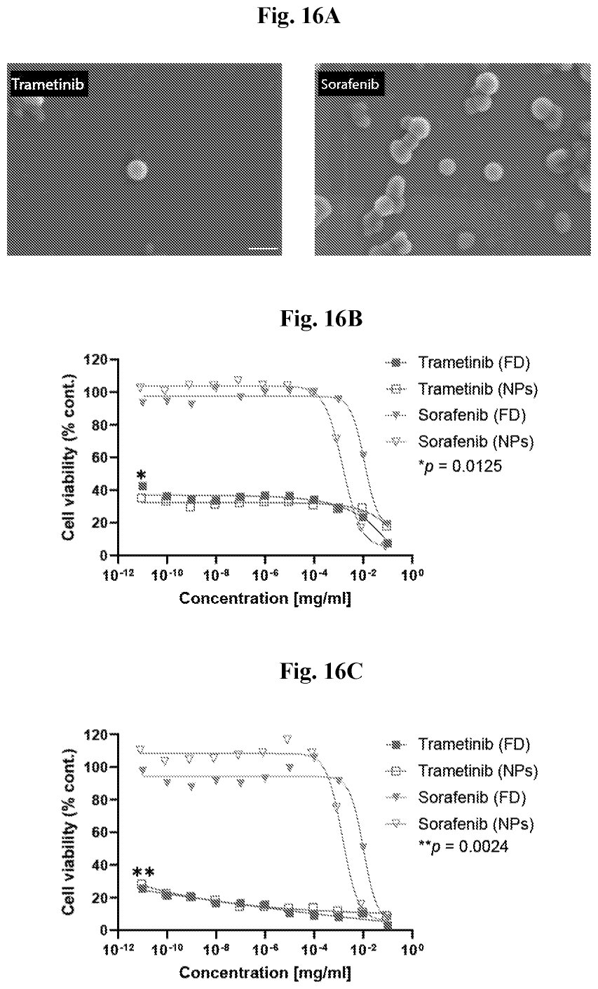

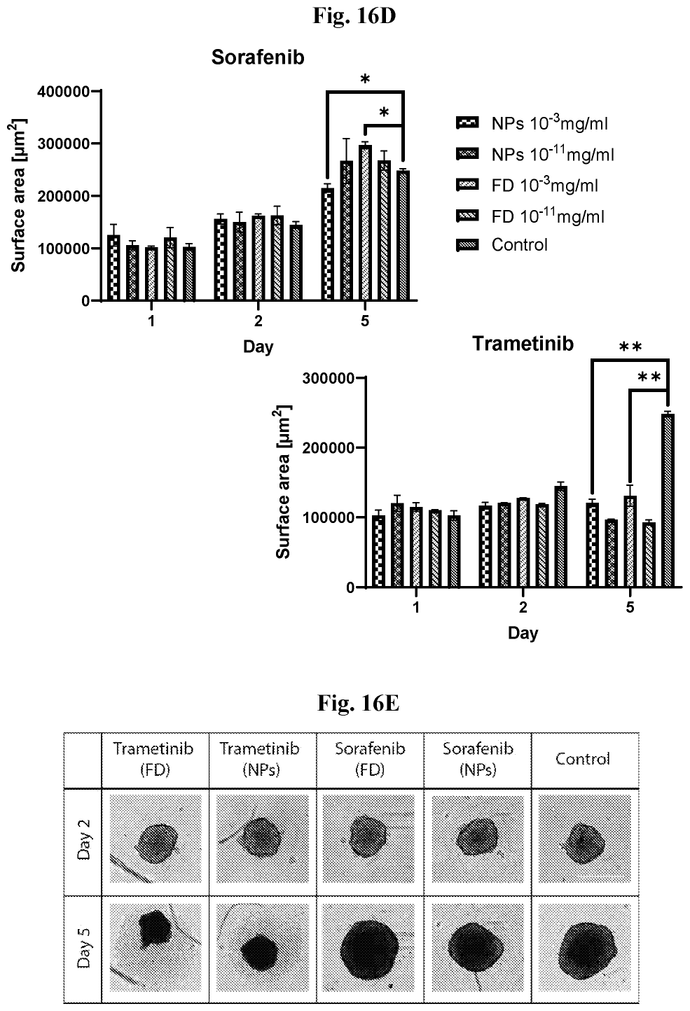

Both formulations were imaged in HR-SEM (FIG. 16A) and demonstrated that the nanoparticles are spherical; however, some aggregates are visible. The average trametinib nanoparticles size according to the SEM images is 68 nm, and according to DLS measurements is 130±7 nm. While for sorafenib nanoparticles, the DLS and SEM measurements are more in agreement—74 nm and 61 nm, respectively. Since particles are expected to shrink in size during the SEM sample preparation process, these results are logical.

The results shown in FIGS. 16B-16C are normalized to the encapsulation efficiency of the nanoparticles, which according to the UHPLC measurements the encapsulation efficiency for trametinib is 90% and approximately 80% for sorafenib. In 2D as well as in the 3D cell cultures the trend is very clear, trametinib is much more efficacious in killing this cell line (P<0.05). Moreover, this trend and the drugs' efficacy maintain when encapsulating them in nanoparticles. After several days of incubation with the treatment, we noticed differences in the morphology of the spheroids that derive from their viability. The spheroids grew in the non-treated wells or wells where the treatment was not effective enough but shrunk or maintained their size in other wells (FIGS. 16D-16E).

### REFERENCES

Liu, T. et al. Synthesis and characterization of a glycine-modified heptamethine indocyanine dye for in vivo cancer-targeted near-infrared imaging. Drug Des. Devel. Ther. 8, 1287 (2014a).
Liu, Y., Ai, K., Lu, L. Polydopamine and its derivative materials: synthesis and promising applications in energy, environmental, and biomedical fields. Chem. Rev. 114, 5057-5115 (2014b).
Narayanan, N., Patonay, G. A new method for the synthesis of heptamethine cyanine dyes: synthesis of new near-infrared fluorescent labels. J. Org. Chem 60, 2391-2395 (1995).
Prostota, Y., Coelho, P. J. Cationic 3H-indolium dyes by ring-opening of benzo[1,3]oxazine. Dyes and Pigments, 98(1), 93-99 (2013).
Ravfogel, S., Taub-Tabib, H., Goldberg, Y. Neural Extractive Search. 210-217 (2021) doi:10.18653/v1/2021.acl-demo.25.
Shamay, Y. et al. Quantitative self-assembly prediction yields targeted nanomedicines. Nat. Mater. 17, 361-368 (2018).
Taub-Tabib, H. et al. Interactive extractive search over biomedical corpora. arXiv preprint arXiv:2006.04148 (2020).
Wang, B., Liu, S., Liu, X., Hu, R., Qin, A., Tang, B. Z. Aggregation-induced emission materials that aid in pharmaceutical research. Adv Healthc Mater, 10(24), (2021).
Wei, Q., Zhang, F., Li, K., Li, B., Zhao, C. Oxidant-induced dopamine polymerization for multifunctional coatings. Polym Chem, 1(9), 1430-1433 (2010).
Wu, T., Huang, J., Yan, Y. Self-assembly of aggregation-induced-emission molecules. Chem Asian J, 14(6), 730-750 (2019).
Würthner, F. Aggregation-induced emission (AIE): a historical perspective. Angew Chem Int Ed Engl, 59(34), 14192-14196 (2020).
Yang, M., Zhou, H., Zhang, Y., Hu, Z., Niu, N., Yu, C. Controlled synthesis of polydopamine: A new strategy for highly sensitive fluorescence turn-on detection of acetylcholinesterase activity. Microchimica Acta, 185(2), 1-7 (2018).
Zhang, C. et al. Dopamine-triggered one-step polymerization and codeposition of acrylate monomers for functional coatings. ACS Appl Mater Interfaces, 9(39), 34356-34366 (2017).
Zhang, Y., Zhao, Y., Xia, S., Tao, L., Wei, Y. A facile preparation of mussel-inspired poly(dopamine phosphonate-co-PEGMA)s via a one-pot multicomponent polymerization system. Macromol Rapid Commun, 41(2), 1900533 (2020).

1. A composition comprising a mixture of oligomers obtained by exposing an indolium-based monomer of formula I, 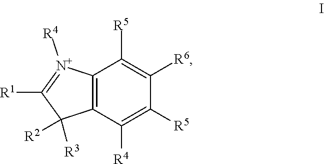
wherein:
R¹, R², and R³ each independently is (C₁-C₆)alkyl optionally interrupted with one or more heteroatoms selected from the group consisting of O, N, and S;
R⁴ is (C₁-C₁₂)alkyl optionally interrupted with one or more heteroatoms selected from the group consisting of O, N, and S, substituted with one or more groups each independently selected from the group consisting of —SO₃⁻, —COO⁻, —PO₃⁻², —OH, —NH₂, —N⁺(R′)₃, phosphocholine (—OPO₃⁻—(CH₂)₂—N⁺(CH₃)₃), and acetoxyethyl phospocholine (—CH₂—C(O)—O—(CH₂)₂—OPO₃⁻—(CH₂)₂—N⁺(CH₃)₃), wherein R′ each independently is (C₁-C₆)alkyl, or (C₃-C₇)cycloalkyl, or two of the R's together with the nitrogen atom to which they are attached form a 5-9 membered ring;
R⁵, R⁶, R⁷, and R⁸ each independently is selected from the group consisting of H, —OH, (C₁-C₆)alkyl, —O—(C₁-C₆)alkyl, —CO(C₁-C₆)alkyl, —COO(C₁-C₆)alkyl, —N(R⁹)₂, —CON(R⁹)₂, —S—(C₁-C₆)alkyl, and —SH; or two adjacent of R⁵, R⁶, R⁷, and R⁸ together with the carbon atoms to which they are attached form (C₆-C₁₄)aryl or 5- to 14-membered heteroaryl, optionally substituted by one or more groups each independently selected from the group consisting of halogen, —OH, (C₁-C₆)alkyl, —O—(C₁-C₆)alkyl, —CO(C₁-C₆)alkyl, —COO(C₁-C₆)alkyl, —N(R⁹)₂, —CON(R⁹)₂, —S—(C₁-C₆)alkyl, and —SH, and the other of R⁵, R⁶, R⁷, and R⁸ each independently is selected from the group consisting of H, —OH, (C₁-C₆)alkyl, —O—(C₁-C₆)alkyl, —CO(C₁-C₆)alkyl, —COO(C₁-C₆)alkyl, —N(R₉)₂, —CON(R⁹)₂, —S—(C₁-C₆)alkyl, and —SH; and
R⁹ each independently is H, halogen, (C₁-C₆)alkyl, (C₃-C₁₁)cycloalkyl, (C₅-C₁₁)cycloalkenyl, heterocyclyl, aryl, heteroaryl, or two R⁹'s together with the nitrogen atom to which they are attach form a 5- to 7-membered ring,
optionally together with an additional monomer selected from the group consisting of dopamine, L-dopa, norepinephrine, serotonin, and a mixture thereof, to a basic buffer.

2. The composition of claim 1, wherein R¹, R², and R³ each independently is (C₁-C₆)alkyl.

3. The composition of claim 2, wherein R¹, R², and R³ are identical.

4. The composition of claim 3, wherein R¹, R², and R³ each is methyl.

5. The composition of claim 1, wherein R⁴ is (C₁-C₆)alkyl substituted with one or more —SO₃⁻, phosphocholine, or acetoxyethyl phospocholine groups.

6. The composition of claim 5, wherein R⁴ is a linear (C₁-C₆)alkyl substituted with a sole —SO₃⁻, phosphocholine, or acetoxyethyl phospocholine group at the omega position thereof.

7. The composition of claim 6, wherein R⁴ is —(CH₂)₄—SO₃—, —(CH₂)₃—OPO₃⁻—(CH₂)₂—N⁺(CH₃)₃, or —(CH₂)₄—C(O)—O—(CH₂)₂—OPO₃⁻—(CH₂)₂—N⁺(CH₃)₃.

8. The composition of claim 1, wherein R⁵, R⁶, R⁷, and R⁸ each independently is H, or (C₁-C₆)alkyl; or R⁵ and R⁶ each independently is H, or (C₁-C₆)alkyl, and R⁷ and R⁸ together with the carbon atoms to which they are attached form an optionally substituted (C₆-C₁₄)aryl.

9. The composition of claim 8, wherein R⁷ and R⁸ together with the carbon atoms to which they are attached form phenyl.

10. The composition of claim 1, wherein:
R¹, R², and R³ each independently is (C₁-C₆);
R⁴ is (C₁-C₆)alkyl substituted with one or more —SO₃⁻, phosphocholine, or acetoxyethyl phospocholine groups; and
R⁵, R⁶, R⁷, and R⁸ each independently is H, or (C₁-C₆)alkyl; or R⁵ and R⁶ each independently is H, or (C₁-C₆)alkyl, and R⁷ and R⁸ together with the carbon atoms to which they are attached form an optionally substituted (C₆-C₁₄)aryl.

11. The composition of claim 10, wherein:
R¹, R², and R³ are identical;
R⁴ is a linear (C₁-C₆)alkyl substituted with a sole —SO₃⁻, phosphocholine, or acetoxyethyl phospocholine group at the omega position thereof; and
R⁷ and R⁸ together with the carbon atoms to which they are attached form phenyl.

12. The composition of claim 11, wherein:
(i) R¹, R², and R³ each is methyl; R⁴ is —(CH₂)₄—SO₃⁻; and R⁵, R⁶, R⁷, and R⁸ each is H (2,3,3-trimethyl-1-(4-sulfobutyl)indolium inner salt, also referred to herein as In783);
(ii) R¹, R², and R³ each is methyl; R⁴ is —(CH₂)₄—SO₃⁻; R⁵ and R⁶ each is H; and R⁷ and R⁸ together with the carbon atoms to which they are attached form phenyl (1,1,2-trimethyl-3-(4-sulfobutyl)benz[e]indolium inner salt, also referred to herein as In820);
(iii) R¹, R², and R³ each is methyl; R⁴ is —(CH₂)₃—OPO₃⁻—(CH₂)₂—N⁺(CH₃)₃; R⁵ and R⁶ each is H; and R⁷ and R⁸ together with the carbon atoms to which they are attached form phenyl (3-(1,1,2-trimethyl-1H-benzo[e]indol-3-ium-3-yl)propyl phosphocholine); or
(iv) R¹, R², and R³ each is methyl; R⁴ is —(CH₂)₄—C(O)—O—(CH₂)₂—OPO₃⁻—(CH₂)₂—N⁺(CH₃)₃—; R⁵ and R⁶ each is H; and R⁷ and R⁸ together with the carbon atoms to which they are attached form phenyl (2-(2-(1,1,2-trimethyl-1H-benzo [e]indol-3-ium-3-yl)acetoxy)ethyl phosphocholine).

13. The composition of claim 1, wherein said basic buffer is tris(hydroxymethyl)aminomethane (TRIS) buffer, 4-(2-hydroxyethyl)-1-piperazineethanesulfonic acid (HEPES) buffer, 3-[N-tris(hydroxymethyl)methylamino]-2-hydroxypropanesulfonic acid (TAPSO) buffer, N-[tris(hydroxymethyl)methyl]glycine (Tricine) buffer, 2-(bis(2-hydroxyethyl)amino)acetic acid (Bicine) buffer, [tris(hydroxymethyl)methylamino]propanesulfonic acid (TAPS) buffer, a borate-based buffer, a tetraborate-based buffer, or a bicarbonate-based buffer, potassium bicarbonate buffer, ammonium bicarbonate buffer, triethylammonium bicarbonate buffer, and carbonate bicarbonate buffer.

14. The composition of claim 13, wherein said buffer is a bicarbonate-based buffer.

15. The composition of claim 1, wherein: (i) said indolium-based monomer has been exposed to said basic buffer at a temperature of from about 18° C. to about 150° C.; (ii) said indolium-based monomer has been exposed to said basic buffer for at least 2 hours; and/or (iii) a concentration of said indolium-based monomer is from about 1 mg/ml to about 100 mg/ml.

16. The composition of claim 1, obtained by:
(a) exposing 1,1,2-trimethyl-3-(4-sulfobutyl) benz[e]indolium inner salt to a bicarbonate-based buffer; or
(b) exposing 1,1,2-trimethyl-3-(4-sulfobutyl) benz[e]indolium inner salt, together with an additional monomer selected from the group consisting of dopamine, L-dopa, norepinephrine, serotonin, and a mixture thereof, to a bicarbonate-based buffer.

17. (canceled)

18. The composition of claim 16, wherein:
said additional monomer is dopamine, L-dopa, or a mixture thereof; or
said additional monomer is dopamine or serotonin, and a molar ratio between the 1,1,2-trimethyl-3-(4-sulfobutyl)benz[e]indolium inner salt, and said dopamine or serotonin is about 2:1, respectively.

19. The composition of claim 18, wherein said additional monomer is a mixture of dopamine and L-dopa, and the molar ratio between the 1,1,2-trimethyl-3-(4-sulfobutyl) benz[e]indolium inner salt, said dopamine, and said L-dopa is about 1:1:1, respectively.

20. (canceled)

21. The composition of claim 1, further comprising a hydrophobic substance.

22. The composition of claim 21, in the form of (a) a suspension of nanoparticles, each comprising said mixture of oligomers and said hydrophobic substance, in an aqueous liquid; or (b) a powder.

23. The composition of claim 22, wherein a size of said nanoparticles ranges from about 20 nm to about 400 nm.

24. (canceled)

25. The composition of claim 34, wherein said hydrophobic substance is a drug selected from the group consisting of an anticancer (chemotherapeutic) drug, antifungal drug, antibacterial drug, antiviral drug, immunosuppressive drug, anti-hyperlipidemic drug, nonsteroidal anti-inflammatory drug, cardiac drug, neurological drug, psychoactive drug, drug of abuse, alkaloid, antibiotic, bioactive peptide, steroid, steroid hormone, selective estrogen receptor modulator (SERM), 5-alpha reductase inhibitor, peptide hormone, interferon, interleukin, narcotic, nucleic acid, pesticide, and prostaglandin.

26. The composition of claim 25, wherein: (i) said anticancer drug is a protein kinase inhibitor; angiogenesis and myeloma cell growth inhibitor; an antiandrogen medication; an anthracycline antibiotic; a B-cell lymphoma 2 inhibitor; a taxane-based drug; an antitumor antibiotic; a benzamide histone deacetylase inhibitor; a cancer cell stemness inhibitor; a flavagline; a Hedgehog signaling pathway targeting agent; a transforming growth factor beta (TGF-β) inhibitor; a poly ADP ribose polymerase (PARP) inhibitor; a retinoid-based drug; a topoisomerase inhibitor; an epidermal growth factor receptor (EGFR) inhibitor; a mammalian target of rapamycin (mTOR) inhibitor; a proteasome inhibitor; or a nonsteroidal antiandrogen (NSAA) medication; (ii) said antibacterial drug is a soluble adenylyl cyclase inhibitor; (iii) said anti-hyperlipidemic drug is probucol; (iv) said 5-alpha reductase inhibitor is dutasteride; (v) said nonsteroidal anti-inflammatory drug is a cyclooxygenase-2 inhibitor; (vi) said selective estrogen receptor modulator is ospemifene; or (vii) said immunosuppressive drug is a calcineurin inhibitor.

27. The composition of claim 34, wherein said hydrophobic substance is a hydrophobic drug comprising a 2-(N-anilino)pyrimidine group coupled to π-conjugated system directly or via a nitrogen atom.

28. The composition of claim 21, formulated for enteral administration, parenteral administration, inhalation.

29-31. (canceled)

32. The composition of claim 13, wherein said borate-based buffer is sodium borate; said tetraborate-based buffer is sodium tetraborate or disodium tetraborate; or said bicarbonate-based buffer is sodium bicarbonate buffer, potassium bicarbonate buffer, ammonium bicarbonate buffer, triethylammonium bicarbonate buffer, or carbonate bicarbonate buffer.

33. The composition of claim 15, wherein: (i) said indolium-based monomer has been exposed to said basic buffer at a temperature of about 90° C.; (ii) said indolium-based monomer has been exposed to said basic buffer for from about 4 hours to about 36 hours; and/or (iii) the concentration of said indolium-based monomer is from about 2 mg/ml to about 10 mg/ml.

34. The composition of claim 21, wherein said hydrophobic substance is a drug or a dietary supplement.

35. The composition of claim 26, wherein: (i) said protein kinase inhibitor is selected from the group consisting of nilotinib, sorafenib, staurosporine, midostaurin, ibrutinib, trametinib, regorafenib, ponatinib, afatinib, pazopanib, rociletinib, dasatinib, ceritinib, ulixertinib, cabozantinib, nintedanib, selumetinib, thiazovivin, osimertinib, defactinib, idelalisib, taselisib, mubritinib, infigratinib, duvelisib, lapatinib, and avapritinib; said angiogenesis and myeloma cell growth inhibitor is pomalidomide; said antiandrogen medication is bicalutamide; said anthracycline antibiotic is valrubicin; said B-cell lymphoma 2 inhibitor is navitoclax; said taxane-based drug is paclitaxel or docetaxel; said antitumor antibiotic is tanespimycin; said benzamide histone deacetylase inhibitor is mocetinostat; said cancer cell stemness inhibitor is napabucasin; said flavagline is rocaglamide; said Hedgehog signaling pathway targeting agent is vismodegib; said transforming growth factor beta (TGF-β) inhibitor is galunisertib; said poly ADP ribose polymerase (PARP) inhibitor is talazoparib; said retinoid-based drug is alitretinoin; said topoisomerase inhibitor is selected from the group consisting of camptothecin, etoposide, and irinotecan; said epidermal growth factor receptor (EGFR) inhibitor is gefitinib or erlotinib; said mammalian target of rapamycin (mTOR) inhibitor is everolimus or rapamycin; said proteasome inhibitor is carfilzomib; said nonsteroidal antiandrogen (NSAA) medication is enzalutamide; said soluble adenylyl cyclase inhibitor is bithionol; said cyclooxygenase-2 inhibitor is celecoxib; or said calcineurin inhibitor is tacrolimus or cyclosporine.
